(19) United States
(12) Patent Application Publication
(10) Pub. No.: US 2024/0247066 A1
(43) Pub. Date: Jul. 25, 2024

(54) STRUCTURE BASED ISOLATION OF PMHC-RESTRICTED ANTIBODIES

(71) Applicant: The Board of Trustees of the Leland Stanford Junior University, Stanford, CA (US)

(72) Inventors: Kenan Christopher Garcia, Menlo Park, CA (US); Daisuke Nishimiya, Redwood City, CA (US); Xinbo Yang, Palo Alto, CA (US)

(21) Appl. No.: 18/560,003

(22) PCT Filed: May 11, 2022
(86) PCT No.: PCT/US2022/028809

§ 371 Date: Nov. 9, 2023

Related U.S. Application Data
(60) Provisional application No. 63/187,735, filed on May 12, 2021.

(51) Int. Cl. C07K 16/28; A61K 38/00; C07K 14/725; G01N 33/68

(52) U.S. Cl. CPC C07K 16/2833; C07K 14/7051; G01N 33/6845; A61K 38/00; C07K 2317/622; C07K 2319/30

(57) ABSTRACT

Compositions and methods are provided for the development and screening of antibody-based binding regions (ABR) that mimic T cell receptor specificity (TCRm). The TCRm-ABR of the disclosure are developed to specifically bind to the combination of an MHC antigen and peptide, and to substantially lack binding to the MHC in the absence of the cognate antigen.

### CROSS REFERENCE TO RELATED APPLICATION

The present application claims the benefit of and priority to U.S. Provisional Patent Application No. 63/187,735, filed May 12, 2021, the entire disclosure of which is hereby incorporated by reference in its entirety.

### INCORPORATION BY REFERENCE OF SEQUENCE LISTING PROVIDED AS A TEXT FILE

A Sequence Listing is provided here within in a text file, (S21-035_STAN-1830WO_Seq_Listing_ST25.txt), created on May 11, 2022, and having a size of 22,000 bytes. The contents of the text file are incorporated herein by reference in its entirety.

### BACKGROUND

Antigens, including tumor antigens, are presented by MHC molecules on tumor cells. T cell receptors (TCR) normally see the MHC-bound peptide (pMHC) to mount an immune response. Antibodies that can recognize the pMHC structure in a manner analogous to the TCR would be highly desirable agents for immunotherapy, because they could enable very selective targeting of tumor cells expressing the particular antigen presented by the MHC. However, TCRs bind to pMHC molecules in a structurally complex interaction where the TCR binds to both MHC and peptide in a peptide specific manner, referred to as MHC restriction.

Previously, antibodies that mimic the TCR interaction with peptide and MHC, referred to as TCRm, have been selected by a cumbersome process. These TCRm Abs have the ability to bind to the peptide and MHC molecules like a TCR, but they have been clinically limited by binding to the MHC alone, giving rise to off-target reactivity.

Methods and compositions for improved screening platforms that identify antibodies with specific pMHC binding are of interest, and are addressed herein.

### SUMMARY

Compositions and methods are provided for the development and screening of antibody-based binding regions (ABR) that mimic T cell receptor specificity (TCRm). The TCRm-ABR of the disclosure are developed to specifically bind to the combination of an MHC antigen and peptide, and to substantially lack binding to the MHC in the absence of the cognate antigen.

Compositions and methods are provided for the identification of ABRs that have affinity for an MHC-peptide sequences of interest. In such methods, a structural analysis is performed on the interaction between an initial ABR that binds to an initial MHC protein of interest in combination with an initial peptide. The structural analysis is used to pinpoint the specific CDR residues that contact the peptide portion of the antigen. An library of polynucleotide sequences encoding ABRs is generated by randomization of the CDR residues thus identified. The number of contact residues to be randomized is determined by the ABR structure, and may vary from about 7 to about 10 residues.

The library of ABRs may be formatted as single chain variable regions (scFv) operably linked to an expression vector. The ABR coding sequence may be fused to a domain that allows the polypeptide to be tethered to a cell surface, including without limitation yeast Aga2, or is a transmembrane domain that allows display on a cell surface. In some embodiments the library comprises a diversity of at least 10⁷ different sequences, at least 5×10⁷ different sequences, at least 10⁸ different sequences, and may comprise at least 5×10⁸ different sequences. For screening, the library is introduced into a suitable host cell that expresses the encoded polypeptide, which host cells include, without limitation, yeast cells.

A soluble MHC-peptide complex, optionally a multimerized complex to enhance binding, is used to select for host cells expressing an ABR that specifically binds to the complex. The MHC component is generally the initial MHC protein, but the peptide component may be distinct from the initial peptide. Iterative rounds of selection are performed, i.e. the cells that are selected in the first round provide the starting population for the second round, etc. until the selected population has a signal above background, usually at least three and more usually at least four rounds of selection are performed. Polynucleotides encoding the final selected ABR from the library can be reformatted into various configurations, e.g. scFv, intact antibody, etc., and may be may be formatted into a therapeutic agent that activates cytolytic pathways, e.g. by fusion to an Fc effector region, as a CAR-T construct, as a CD3-based bispecific T-cell engager, and the like. For autoimmune indications the antigen binding region may be formatted to block interactions without activating immune cytotoxic pathways.

Various peptides are useful with the technology when complexed to an MHC. Viral peptides can be utilized from any virus of interest, including without limitation RNA viruses, such as coronavirus, e.g. SARS-CoV1, SARS-CoV2, MERS-CoV, etc. Cancer neoantigens are also of interest, which may be over-expressed, expressed in non-normal tissues, comprise antigenic mutations, and the like. (see for example Schumacher et al. (2019) Annu Rev Immunol 37:173-200). Autoantigens are also of interest, e.g. antigens expressed in MS, IDDM, RA, etc.

MHC proteins of interest include any of the mammalian MHC proteins. Human HLA proteins are of interest, particularly HLA Class I proteins, e.g. human HLA-A, HLA-B, HLA-C. In some embodiments an allele of HLA-A or HLA-B is screened. As will be appreciated by one with skill in the art, the HLA locus is highly polymorphic and a large number of sequence variants are known and described in the art, including without limitation any of the HLA-A*01, HLA-A*02, up to HLA-A*80 alleles and serotypes thereof; and the HLA-B*07, HLA-B*08 up to HLA-B*83 and serotypes thereof. MHC sequences used for screening purposes typically comprise the peptide binding region, e.g. the alpha 1 and alpha 2 domains, or the portion of those domains required to form a peptide binding complex. The MHC sequences for screening may be a naturally occurring sequence, or may be a designed consensus sequence to provide pan-allelic specificity. For some embodiments HLA Class II proteins are of interest, e.g. HLA-DPA1, HLA-DPB1, HLA-DQA1, HLA-DQB1, HLA-DRA, and HLA-DRB1.

In an embodiment, an ABR library is provided for ABR with specificity for the human HLA protein HLA-A2, with an initial ABR comprising the CDR sequences set forth in SEQ ID NO:1-6. The randomized CDR sequences may encode the sequences set forth in SEQ ID NO:7 (H-CDR3); SEQ ID NO:8 (L-CDR1); and SEQ ID NO:9 (L-CDR3). Optionally, the ABRs utilize the framework sequences of SEQ ID NO:86 and SEQ ID NO:87.

In an embodiment, an ABR specific for human HLA-A2 and NY-ESO1 peptide, e.g. SEQ ID NO:79, is provided, where the ABR comprises the CDR sequences SEQ ID NO:1 (H-CDR1); SEQ ID NO:2 (H-CDR2); and SEQ ID NO:5 (L-CDR2) appropriately combined with affinity-selected CDR sequences selected from SEQ ID NO:10-12; SEQ ID NO:10, 4 and 15; SEQ ID NO:16-18.

In an embodiment, an ABR specific for human HLA-A2 and MART1 peptide, e.g. SEQ ID NO:80, is provided, where the ABR comprises the CDR sequences SEQ ID NO:1 (H-CDR1); SEQ ID NO:2 (H-CDR2); and SEQ ID NO:5 (L-CDR2) appropriately combined with affinity-selected CDR sequences selected from SEQ ID NO:19-21; SEQ ID NO:22-24.

In an embodiment, an ABR specific for human HLA-A2 and gp100 peptide, e.g. SEQ ID NO:81, is provided, where the ABR comprises the CDR sequences SEQ ID NO:1 (H-CDR1); SEQ ID NO:2 (H-CDR2); and SEQ ID NO:5 (L-CDR2) appropriately combined with affinity-selected CDR sequences selected from SEQ ID NO:10 and 26-27.

In an embodiment, an ABR specific for human HLA-A2 and gp100 peptide, e.g. SEQ ID NO:82, is provided, where the ABR comprises the CDR sequences SEQ ID NO:1 (H-CDR1); SEQ ID NO:2 (H-CDR2); and SEQ ID NO:5 (L-CDR2) appropriately combined with affinity-selected CDR sequences selected from SEQ ID NO:10 and 26-27; or SEQ ID NO:10 and 29-30.

In an embodiment, an ABR specific for human HLA-A2 and KRAS peptide, e.g. SEQ ID NO:83, is provided, where the ABR comprises the CDR sequences SEQ ID NO:1 (H-CDR1); SEQ ID NO:2 (H-CDR2); and SEQ ID NO:5 (L-CDR2) appropriately combined with affinity-selected CDR sequences selected from SEQ ID NO:19 and 32-33; or SEQ ID NO:19 and 35-36.

In an embodiment, an ABR library is provided for ABR with specificity for the mouse H-2K^{b} protein, with an initial ABR comprising the CDR sequences set forth in SEQ ID NO:37-42. The randomized CDR sequences may encode the sequences set forth in SEQ ID NO:43 (H-CDR1), SEQ ID NO:44 (H-CDR2), SEQ ID NO:45 (H-CDR3) and SEQ ID NO:46 (L-CDR3). Optionally, the ABRs utilize the framework sequences of SEQ ID NO:84 and SEQ ID NO:85.

In an embodiment, an ABR specific for mouse H-2K^{b} and TRP2 peptide is provided, where the ABR comprises the CDR sequences SEQ ID NO:40 (L-CDR1); and SEQ ID NO:41 (L-CDR2) appropriately combined with affinity-selected CDR sequences selected from SEQ ID NO:47-50; SEQ ID NO:51-54, SEQ ID NO:55-58 or SEQ ID NO:55 and 60-62.

In an embodiment, a ABR that provides for specific binding to an antigenic peptide in an MHC context as described above, is covalently linked, e.g. conjugated or fused, to an effector polypeptide, e.g. an immunoglobulin effector sequence, for example an Fc sequence, etc., in a chimeric antigen receptor (CAR), in a CD3-based bispecific T-cell engager, and the like, to generate a therapeutic entity. A polypeptide may be labeled with a detectable label, immobilized on a solid phase and/or conjugated with a heterologous compound, e.g. toxin, etc.

In some embodiments, an engineered cell is provided, e.g. an engineered T cell, in which the cell has been modified by introduction of a ABR coding sequence, e.g. a chimeric antigen receptor (CAR), etc. The engineered cell can be provided in a unit dose for therapy, and can be allogeneic, autologous, etc., with respect to an intended recipient. Introduction of the coding sequence can be performed in vivo or in vitro, using any appropriate vector, e.g., viral vectors, integrating vectors, and the like.

In some embodiments, a therapeutic method is provided, particularly relating to the elimination of virus infected cells or cancer cells, by administering to an individual an effective dose of a therapeutic entity or cell expressing a therapeutic entity as described above.

In some embodiments, a vector comprising a polynucleotide sequence encoding a polypeptide comprising an ABR sequence is provided, where the coding sequence is operably linked to a promoter active in the desired cell. In some embodiments, the promoter may be constitutive or inducible. Various vectors are known in the art and can be used for this purpose, e.g. viral vectors, plasmid vectors, minicircle vectors, etc. which vectors can be integrated into the target cell genome, or can be episomally maintained. The vector may be provided in a kit.

In some embodiments, a kit is provided for the identification of protein sequences that bind to an MHC-peptide complex of interest. Such a kit may comprise a library of polynucleotides as disclosed herein, where a diverse set of sequences is provided, e.g. at least 10⁶, at least 10⁷, more usually at least 10⁸ different sequences are present in the library. The polynucleotide library can be provided as a population of transfected cells, or as an isolated population of nucleic acids. Reagents for labeling and multimerizing an MHC-peptide complex can be included. In some embodiments the kit will further comprise a software package for analysis of a sequence database.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Schematic depicting the generation of structure-based re-purposing of a TCR mimic antibody.

FIGS. 2A-2B. Design and construction of mouse TCR mimic scFv Library. (a) the amino acid residues of TCR like antibody for OVA/H-2K^{b} (PDB; 3cvh) in close contact (4 Å) to a peptide displayed on H-2K^{b} were calculated and picked up by Collaborative Computational Project 4 (CCP4) and PyMOL. The variable light chain domain (V_{L}) was colored cyan, the variable heavy chain domain (V_{H}) was colored purple, and MHC was colored green. Contact residues of antibody were colored magenta, and the OVA peptide (SEQ ID NO:88) was colored red. By introducing randomized amino acids excluding Cys into the selected 9 residues, a mouse TCRm scFv yeast library at the diversity of 5×10⁸ was created. (b) Schematic representation of the construct for the TCR mimic scFv yeast display library.

FIGS. 3A-3B. The CDR sequences for mouse TCR mimic Ab; a) heavy chain (SEQ ID NO:37-39 for CDR1-CDR3 respectively) and b) light chain (SEQ ID NO:40-42 for CDR1-CDR3 respectively). Interacting residues of an antibody with the OVA peptide of pMHC were marked as “P” and interacting residues with MHC were marked as “M”. Nine interacting residues on CDRs were selected, and the randomized amino acids (except for Cys) were introduced into the amino acid residues which were highlighted in pink to make a TCR mimic scFv yeast library; (a) heavy chain and (b) light chain.

FIG. 4. Yeast display screening of mouse TCRm scFv Library for Trp2/H-2K^{b}. Yeast display selection against mouse Trp2/H-2K′ was performed using mouse TCR mimic scFv library. Specific enrichment for binders was observed after 4 rounds of selection. The panels indicate tetramer pMHC staining on the Y axis and HA-tag staining of the scFv on the X-axis.

FIGS. 5A-5B. Binding characteristics of individual yeast clones expressing scFv for Trp2/H-2K^{b}. The top 4 candidates were selected via monomer and tetramer pMHC staining. (a) The individual yeast clones bound to monomer Trp2/H-2K^{b} in a dose-dependent manner. (b) Yeast clones specifically bound to 100 nM of Trp2/H-2K^{b}, compared with 1,000 nM of OVA/H-2K^{b}. Data are representative of two independent experiments.

FIG. 6. Surface plasmon resonance of mouse TCRm Abs binding to Trp2/H-2K^{b}. The binding affinities of mouse TCR mimic scFvs and IgGs were measured by biacoreT100 at room temperature.

FIG. 7. Sequences of mouse TCRm Abs binding TRP2/H-2K^{b}

FIGS. 8A-8D. Trp2 peptide-pulsed cells binding assays. Purified TCRm scFv #13 (a) and IgG #13 (b) specifically bound to Trp2 peptide-pulsed EL4 cells in a dose-dependent manner. (c and d) IgG2a #13 specifically bound to Trp2 peptide-pulsed B16F10 cells treated with IFN-gamma. Data are mean±SD.

FIGS. 9A-9D. ADCC activities of mouse TCRm Ab for Trp2/H-2K^{b}. The mIgG2a #13 DE mutant showed ADCC activity against Trp2 peptide-pulsed B16F10 cells that were treated with IFN-gamma. A) ADCC was not seen with mIgG2a wildtype or LALAPG mutants in the presence of 10 μM of Trp2 peptide. B) ADCC induction required both peptide and IFN-gamma treatment. C)_Trp2 peptide dose response effect on ADCC with and without 100 U/mL IFN-gamma D) IFN-gamma dose response effect on ADCC in the presence of 10 μM of Trp2 peptide. Data are mean+SD.

FIGS. 10A-10C. Cytotoxic activities of mouse TCRm Ab-based BiTE for Trp2/H-2K^{b}. (a) Schematic representation of the BiTE construct. Each scFv was composed of immunoglobulin variable heavy chain (V_{H}) and immunoglobulin variable light chain (V_{L}) domains, which were linked by a 15-residue (G₄S)₃ (SEQ ID NO:89) linker. (b) TCRm Ab-based BiTE showed the cytotoxic activity against Trp2 peptide-pulsed B16F10 cells which were treated with IFN-gamma. BiTE also killed B16F10 tumor cells which were un-pulsed with a Trp2 peptide and untreated with IFN-gamma. BiTE showed the cytotoxic activity against a Trp2 peptide-pulsed EL4 cells. The 2C11 anti-CD3e monomeric scFv was used as a negative control. (c) BiTE showed cytotoxic activity against Trp2 peptide-pulsed B16F10 cells in a dose-dependent manner. Data are mean±SD.

FIG. 11. Design and construction of human TCR mimic scFv Library. The amino acid residues of TCR like antibody for NY-ESO1/HLA-A*0201 (PDB; 3gif) in close contact (4 Å) to a peptide displayed on HLA-A*0201 were calculated and identified using Collaborative Computational Project 4 (CCP4) and PyMOL. Contact residues of antibody were colored blue, and NY-ESO1 (SEQ ID NO:79) peptide was colored red. By introducing randomized amino acids excluding Cys into the selected 7 residues, a human TCRm scFv yeast library at the diversity of 2×10⁸ was created.

FIGS. 12A-12B. The CDR sequences for human TCR mimic Ab; a) heavy chain (SEQ ID NO:1-3 for CDR1-CDR3 respectively) and b) light chain (SEQ ID NO:4-6 for CDR1-CDR3 respectively). Interacting residues of an antibody with the NY-ESO1 peptide of pMHC were marked as “P” and interacting residues with MHC were marked as “M”. Seven interacting residues on CDRs were selected, and the randomized amino acids except for Cys were introduced into the amino acid residues which were highlighted in pink to make a TCR mimic scFv yeast library.

FIGS. 13A-13E. Yeast display screening of human TCRm scFv Library for target peptides/HLA-A*0201. Yeast display selections against (a) NY-ESO1₁₅₇₋₁₆₅/HLA-A*0201, (b) MART1₂₆₋₃₅ (A2L)/HLA-A*0201, (c) gp100₂₀₉₋₂₁₈ (T2M)/HLA-A*0201, (d) gp100₂₈₀₋₂₈₈ (A9V)/HLA-A*0201, and (e) KRAS₅₋₁₄/HLA-A*0201 were performed using human TCR mimic scFv library. Specific enrichment for binders was observed after 4 rounds of selection. The panels indicate tetramer pMHC (y-axis) and yeast display staining (x-axis). Yeast clones selected against specific pMHC for four rounds did not cross react with different non-target pMHCs, demonstrating specificity of the selection process.

FIGS. 14A-14D. Binding characteristics of individual yeast clones to monomeric pMHC. The top scFv candidates per target pMHC were selected via monomer and tetramer pMHC staining. The individual yeast clones bound to (a) NY-ESO1₁₅₇₋₁₆₅/HLA-A*0201, (b) MART1₂₆₋₃₅ (A2L)/HLA-A*0201, (c) gp100₂₈₀₋₂₈₈ (A9V)/HLA-A*0201, and (d) KRAS₅₋₁₄/HLA-A*0201 in a dose-dependent manner. Data are mean±SD.

FIGS. 15A-15D. Specificities of individual yeast clones expressing scFv for each target pMHC. Yeast clones for each target pMHC specifically bound to each target pMHC and not to structurally unrelated pMHCs. 100 nM of target pMHCs and 1,000 nM of non-target pMHCs were used for staining each yeast clone. Data are representative of two independent experiments.

FIGS. 16A-16B. Surface plasmon resonance affinity measurements for human TCRm Abs. (a) The binding affinities of human TCR mimic scFvs and IgGs were measured by biacoreT100. (b)_Sensorgrams of hTCRm Abs. *NY WT is a parental scFv and T1 is affinity-maturated scFv for NY WT.

FIG. 17. Sequences of human TCRm Abs for target peptides/HLA-A*0201.

FIGS. 18A-18B. Cell-based binding assays for human TCRm Abs. Purified TCRm IgG1 and scFv MA2 specifically bound to (a) 293 cells expressing single chain MART1₂₆₋₃₅ (A2L)/HLA-A*0201 with the transmembrane domain and (b) MART1₂₆₋₃₅ (A2L) peptide-pulsed A375 cells. Data are mean±SD.

FIG. 19. ADCC assays for hTCRm Ab. The hIgG1 MA2 DLE mutant showed specific ADCC activity against MART1₂₆₋₃₅ (A2L) peptide-pulsed A375 cells in a dose dependent manner.

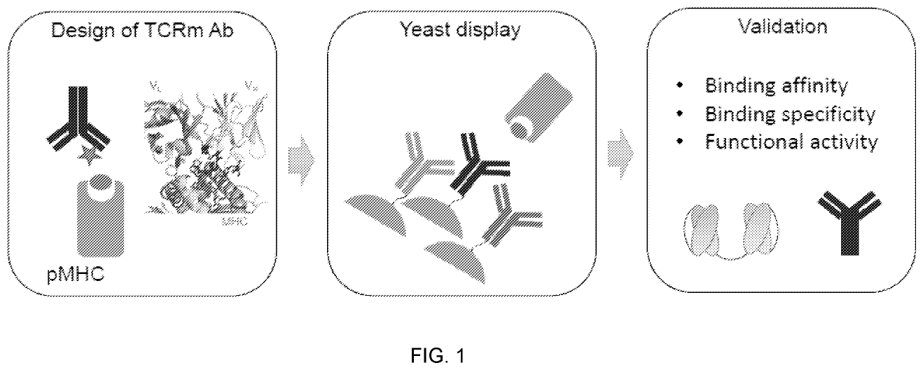

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

MHC Proteins. Major histocompatibility complex proteins (also called human leukocyte antigens, HLA, or the H2 locus in the mouse) are protein molecules expressed on the surface of cells that confer a unique antigenic identity to these cells. MHC/HLA antigens are target molecules that are recognized by T-cells and natural killer (NK) cells as being derived from the same source of hematopoietic reconstituting stem cells as the immune effector cells (“self”) or as being derived from another source of hematopoietic reconstituting cells (“non-self”). Two main classes of HLA antigens are recognized: HLA class I and HLA class II.

The MHC proteins used in the methods of the invention may be from any mammalian or avian species, e.g. primate sp., particularly humans; rodents, including mice, rats and hamsters; rabbits; equines, bovines, canines, felines; etc. Of particular interest are the human HLA proteins. Included in the HLA proteins are the class II subunits HLA-DPα, HLA-DPβ, HLA-DQα, HLA-DQβ, HLA-DRα and HLA-DRβ, and the class I proteins HLA-A, HLA-B, HLA-C, and β2-microglobulin.

The MHC binding domains are typically a soluble form of the normally membrane-bound protein. The soluble form is derived from the native form by deletion of the transmembrane domain. Conveniently, the protein is truncated, removing both the cytoplasmic and transmembrane domains. In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts.

An “allele” is one of the different nucleic acid sequences of a gene at a particular locus on a chromosome. One or more genetic differences can constitute an allele. An important aspect of the HLA gene system is its polymorphism. Each gene, MHC class I (A, B and C) and MHC class II (DP, DQ and DR) exists in different alleles. Current nomenclature for HLA alleles is designated by numbers, as described by Marsh et al.: Nomenclature for factors of the HLA system, 2010. Tissue Antigens 75:291-455, herein specifically incorporated by reference. For HLA protein and nucleic acid sequences, see Robinson et al. (2011), The IMGT/HLA database. Nucleic Acids Research 39 Suppl 1:D1171-6, herein specifically incorporated by reference.

MHC context. The function of MHC molecules is to bind peptide fragments derived from pathogens or aberrant proteins derived from transformed cells, and display them on the cell surface for recognition by the appropriate T cells. Thus, T cell receptor recognition can be influenced by the MHC protein that is presenting the antigen. The term MHC context refers to the recognition by a TCR of a given peptide, when it is presented by a specific MHC protein.

Class II HLA/MHC. In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts.

Class I HLA/MHC. For class I proteins, the binding domains may include the α1, α2 and optionally α3 domain of a Class I allele, including without limitation HLA-A, HLA-B, HLA-C, H-2K, H-2D, H-2L, which are combined with β-microglobulin. In certain specific embodiments, the binding domains are HLA-A2 binding domains, e.g. comprising at least the alpha 1 and alpha 2 domains of an A2 protein. A large number of alleles have been identified in HLA-A2, including without limitation HLA-A*02:01:01:01 to HLA-A*02:478, which sequences are available at, for example, Robinson et al. (2011), The IMGT/HLA database. Nucleic Acids Research 39 Suppl 1:D1171-6. Among the HLA-A2 allelic variants, HLA-A*02:01 is the most prevalent.

Peptide ligands are peptide antigens against which an immune response involving T lymphocyte antigen specific response can be generated. Such antigens include antigens associated with autoimmune disease, infection, cancer neoantigens, foodstuffs such as gluten, etc., allergy or tissue transplant rejection. Antigens also include various microbial antigens, e.g. as found in infection, in vaccination, etc., including but not limited to antigens derived from virus, bacteria, fungi, protozoans, parasites and tumor cells. Tumor antigens include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A; MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, α-fetoprotein; etc.; and self-antigens, e.g. her-2/neu; carcinoembryonic antigen, muc-1, and the like. A benefit of TCRm antigens is that T cells recognize peptides processed and presented on the cell surface, where the protein from which the peptide is derived is not necessarily a cell-surface protein, thereby broadening the number of cancer antigens that can be recognized by antibodies, which normally are not effective against intracellular antigens.

Library. In some embodiments of the invention, a library is provided of polypeptides, or of nucleic acids encoding such polypeptides, where the library comprises ABRs with specific randomized positions in one or more CDR regions, where the randomized positions are selected from identification of peptide contact residues. The number of contact residues to be randomized is determined by the ABR structure, and may vary from about 7 to about 10 residues. The library of ABRs may be formatted as single chain variable regions (scFv) operably linked to an expression vector. The ABR coding sequence may be fused to a domain that allows the polypeptide to be tethered to a cell surface, including without limitation yeast Aga2, or is a transmembrane domain that allows display on a cell surface. In some embodiments the library comprises a diversity of at least 10⁷ different sequences, at least 5×10⁷ different sequences, at least 10⁸ different sequences, and may comprise at least 5×10⁸ different sequences. For screening, the library is introduced into a suitable host cell that expresses the encoded polypeptide, which host cells include, without limitation, yeast cells.

Conventional methods of assembling the coding sequences can be used. In order to generate the diversity of peptide ligands, randomization, error prone PCR, mutagenic primers, and the like as known in the art, are used to create a set of polynucleotides. In various embodiments the library is provided as a purified polynucleotide composition encoding polypeptides, where the population of cells can be, without limitation yeast cells, and where the yeast cells may be induced to express the polypeptide library.

“Suitable conditions” shall have a meaning dependent on the context in which this term is used. That is, when used in connection with binding of a T cell receptor to a pMHC protein, the term shall mean conditions that permit a TCR to bind to a cognate peptide ligand. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

Sequencing platforms that can be used in the present disclosure include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, MA) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058; 3) Applied Biosystems (e.g. SOLiD sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference. Such methods and apparatuses are provided here by way of example and are not intended to be limiting.

Antigen binding region (ABR). As used herein, the term ABR refers to a combination of variable heavy (VH and variable light (VL) polypeptides to associate to form a variable region domain. An ABR is the minimum antibody fragment that contains a complete antigen-recognition and binding site. This region consists of heavy- and one light-chain variable domain in tight, non-covalent association, as a single polypeptide or as a dimer. It is in this configuration that the three CDRS of each variable domain interact to define an antigen-binding site on the surface of the domain. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

An antibody or ABR “which binds” an antigen of interest, is one that binds the antigen with sufficient affinity such that the antibody or binding molecule is useful as a diagnostic and/or therapeutic agent in targeting the antigen, and does not significantly cross-react with other proteins. In such embodiments, the extent of binding of the antibody or other binding molecule to a non-targeted antigen will usually be no more than 10% as determined by fluorescence activated cell sorting (FACS) analysis or radioimmunoprecipitation (RIA).

Antibodies, also referred to as immunoglobulins, conventionally comprise at least one heavy chain and one light, where the amino terminal domain of the heavy and light chains is variable in sequence, hence is commonly referred to as a variable region domain, or a variable heavy (VH) or variable light (VH) domain. The two domains conventionally associate to form a specific binding region, e.g. an ABR, although as well be discussed here, a variety of non-natural configurations of antibodies are known and used in the art.

A “functional” or “biologically active” antibody or antigen-binding molecule is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signaling transduction or enzymatic activity. A functional antibody or other binding molecule may also block ligand activation of a receptor or act as an agonist or antagonist. The capability of an antibody or other binding molecule to exert one or more of its natural activities depends on several factors, including proper folding and assembly of the polypeptide chains.

The term “antibody” herein is used in the broadest sense and specifically covers monoclonal antibodies, polyclonal antibodies, monomers, dimers, multimers, multispecific antibodies (e.g., bispecific antibodies), heavy chain only antibodies, three chain antibodies, single chain Fv, nanobodies, etc., and also include antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain comprising an Fc region, a full length light chain, an intact immunoglobulin molecule comprising Fc region sequences; or an immunologically active portion of any of these polypeptides, i.e., a polypeptide that comprises an antigen binding region (ABR) that immunospecifically binds an antigen of a target of interest or part thereof, such targets including but not limited to, cancer cell or cells that produce autoimmune antibodies associated with an autoimmune disease. The immunoglobulins disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. The immunoglobulins can be derived from any species. In one aspect, the immunoglobulin is of largely human origin.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

Variable regions of interest include the 3 CDR sequences, which may be obtained from available antibodies with the desired specificity, or may be obtained from antibodies developed for this purpose. One of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlan et al. “Identification of specificity-determining residues in antibodies.” Faseb J. 1995; 9:133-139., each of which is herein specifically incorporated by reference.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc.) and human constant region sequences.

An “intact antibody chain” as used herein is one comprising a full length variable region and a full length constant region. An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis (ADCP); and down regulation of cell surface receptors. Constant region variants include those that alter the effector profile, binding to Fc receptors, and the like.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called κ and λ, based on the amino acid sequences of their constant domains.

A “functional Fc region” possesses an “effector function” of a native-sequence Fc region. Exemplary effector functions include C1q binding; CDC; Fc-receptor binding; ADCC; ADCP; down-regulation of cell-surface receptors (e.g., B-cell receptor), etc. Such effector functions generally require the Fc region to be interact with a receptor, e.g. the FcγRI; FcγRIIA; FcγRIIB1; FcγRIIB2; FcγRIIIA; FcγRIIIB receptors, and the low affinity FcRn receptor; and can be assessed using various assays as disclosed, for example, in definitions herein. A “dead” Fc is one that has been mutagenized to retain activity with respect to, for example, prolonging serum half-life, but which does not activate a high affinity Fc receptor. An Fc may also have decreased binding to complement.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

Variant Fc sequences may include three amino acid substitutions in the CH2 region to reduce FcγRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604). Other Fc variants are possible, including without limitation one in which a region capable of forming a disulfide bond is deleted, or in which certain amino acid residues are eliminated at the N-terminal end of a native Fc form or a methionine residue is added thereto. Thus, in one embodiment of the invention, one or more Fc portions of the scFc molecule can comprise one or more mutations in the hinge region to eliminate disulfide bonding. In yet another embodiment, the hinge region of an Fc can be removed entirely. In still another embodiment, the molecule can comprise an Fc variant.

Further, an Fc variant can be constructed to remove or substantially reduce effector functions by substituting, deleting or adding amino acid residues to effect complement binding or Fc receptor binding. For example, and not limitation, a deletion may occur in a complement-binding site, such as a C1q-binding site. Techniques of preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

The Fc may be in the form of having native sugar chains, increased sugar chains compared to a native form or decreased sugar chains compared to the native form, or may be in an aglycosylated or deglycosylated form. The increase, decrease, removal or other modification of the sugar chains may be achieved by methods common in the art, such as a chemical method, an enzymatic method or by expressing it in a genetically engineered production cell line. Such cell lines can include microorganisms, e.g. Pichia Pastoris, and mammalians cell line, e.g. CHO cells, that naturally express glycosylating enzymes. Further, microorganisms or cells can be engineered to express glycosylating enzymes, or can be rendered unable to express glycosylation enzymes (See e.g., Hamilton, et al., Science, 313:1441 (2006); Kanda, et al, J. Biotechnology, 130:300 (2007); Kitagawa, et al., J. Biol. Chem., 269 (27): 17872 (1994); Ujita-Lee et al., J. Biol. Chem., 264 (23): 13848 (1989); Imai-Nishiya, et al, BMC Biotechnology 7:84 (2007); and WO 07/055916). As one example of a cell engineered to have altered sialylation activity, the alpha-2,6-sialyltransferase 1 gene has been engineered into Chinese Hamster Ovary cells and into sf9 cells. Antibodies expressed by these engineered cells are thus sialylated by the exogenous gene product. A further method for obtaining Fc molecules having a modified amount of sugar residues compared to a plurality of native molecules includes separating said plurality of molecules into glycosylated and non-glycosylated fractions, for example, using lectin affinity chromatography (See e.g., WO 07/117505). The presence of particular glycosylation moieties has been shown to alter the function of Immunoglobulins. For example, the removal of sugar chains from an Fc molecule results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. Additional important modifications include sialylation and fucosylation: the presence of sialic acid in IgG has been correlated with anti-inflammatory activity (See e.g., Kaneko, et al, Science 313:760 (2006)), whereas removal of fucose from the IgG leads to enhanced ADCC activity (See e.g., Shoj-Hosaka, et al, J. Biochem., 140:777 (2006)).

In alternative embodiments, antibodies of the invention may have an Fc sequence with enhanced effector functions, e.g. by increasing their binding capacities to FcγRIIIA and increasing ADCC activity. For example, fucose attached to the N-linked glycan at Asn-297 of Fc sterically hinders the interaction of Fc with FcγRIIIA, and removal of fucose by glyco-engineering can increase the binding to FcγRIIIA, which translates into >50-fold higher ADCC activity compared with wild type IgG1 controls. Protein engineering, through amino acid mutations in the Fc portion of IgG1, has generated multiple variants that increase the affinity of Fc binding to FcγRIIIA. Notably, the triple alanine mutant S298A/E333A/K334A displays 2-fold increase binding to FcγRIIIA and ADCC function. S239D/1332E (2X) and S239D/1332E/A330L (3X) variants have a significant increase in binding affinity to FcγRIIIA and augmentation of ADCC capacity in vitro and in vivo. Other Fc variants identified by yeast display also showed the improved binding to FcγRIIIA and enhanced tumor cell killing in mouse xenograft models. See, for example Liu et al. (2014) JBC 289(6):3571-90, herein specifically incorporated by reference.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, an antibody having an Fc region according to this invention can comprise an antibody with or without K447.

“Fv” is the minimum antibody fragment, which contains a complete antigen-recognition and antigen-binding site. The CD3 binding antibodies of the invention comprise a dimer of one heavy chain and one light chain variable domain in tight, non-covalent association; however additional antibodies, e.g. for use in a multi-specific configuration, may comprise a VH in the absence of a VL sequence. Even a single variable domain (or half of an Fv comprising only three hypervariable regions specific for an antigen) has the ability to recognize and bind antigen, although the affinity may be lower than that of two domain binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

“Humanized” forms of non-human (e.g., rodent) antibodies, including single chain antibodies, are chimeric antibodies (including single chain antibodies) that contain minimal sequence derived from non-human immunoglobulin. See, for example, Jones et al, (1986) Nature 321:522-525; Chothia et al (1989) Nature 342:877; Riechmann et al (1992) J. Mol. Biol. 224, 487-499; Foote and Winter, (1992) J. Mol. Biol. 224:487-499; Presta et al (1993) J. Immunol. 151, 2623-2632; Werther et al (1996) J. Immunol. Methods 157:4986-4995; and Presta et al (2001) Thromb. Haemost. 85:379-389. For further details, see U.S. Pat. Nos. 5,225,539; 6,548,640; 6,982,321; 5,585,089; 5,693,761; 6,407,213; Jones et al (1986) Nature, 321:522-525; and Riechmann et al (1988) Nature 332:323-329.

Chimeric antigen receptor (CAR). A CAR is comprised of the general structure where an antigen binding domain, e.g. a ABR sequence disclosed herein, usually provided in an scFv format, is linked to T cell receptor effector functions. The term refers to artificial multi-module molecules capable of triggering or inhibiting the activation of an immune cell. A CAR will generally comprise a ABR sequence as described herein, linker, transmembrane domain and cytoplasmic signaling domain. In some instances, a CAR will include one or more co-stimulatory domains and/or one or more co-inhibitory domains.

A spacer (linker) region links the antigen binding domain to the transmembrane domain. It should be flexible enough to allow the antigen binding domain to orient in different directions to facilitate antigen recognition. The simplest form is the hinge region from an immunoglobulin, e.g. the hinge from any one of IgG1, IgG2a, IgG2b, IgG3, IgG4, particularly the human protein sequences. Alternatives include the CH2CH3 region of immunoglobulin and portions of CD3. For many scFv based constructs, an IgG hinge is effective. In some embodiments the linker comprises the amino acid sequence (G₄S)ₙ (SEQ ID NO:89) where n is 1, 2, 3, 4, 5, etc., and in some embodiments n is 3.

The CAR transmembrane domain (TM) is frequently derived from type I membrane proteins, such as CD3ζ, CD4, CD8, CD28, etc.

A cytoplasmic signaling domain, such as those derived from the T cell receptor ζ-chain, is employed as part of the CAR in order to produce stimulatory signals for T lymphocyte proliferation and effector function following engagement of the chimeric receptor with the target antigen. Endodomains from co-stimulatory molecules may be included in the cytoplasmic signaling portion of the CAR.

The term “co-stimulatory domain”, refers to a stimulatory domain, typically an endodomain, of a CAR that provides a secondary non-specific activation mechanism through which a primary specific stimulation is propagated. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. Nat Rev Immunol (2013) 13(4):227-42, the disclosure of which are incorporated herein by reference in their entirety. Non-limiting examples of suitable co-stimulatory polypeptides include, but are not limited to, 4-1BB (CD137), CD28, ICOS, OX-40, BTLA, CD27, CD30, GITR, and HVEM.

The term “co-inhibitory domain” refers to an inhibitory domain, typically an endodomain, derived from a receptor that provides secondary inhibition of primary antigen-specific activation mechanisms which prevents co-stimulation. Co-inhibition, e.g., T cell co-inhibition, and the factors involved have been described in Chen & Flies. Nat Rev Immunol (2013) 13(4):227-42 and Thaventhiran et al. J Clin Cell Immunol(2012) S12. In some embodiments, co-inhibitory domains homodimerize. A co-inhibitory domain can be an intracellular portion of a transmembrane protein. Non-limiting examples of suitable co-inhibitory polypeptides include, but are not limited to, CTLA-4 and PD-1.

A first-generation CAR transmits the signal from antigen binding through only a single signaling domain, for example a signaling domain derived from the high-affinity receptor for IgE FcεRIγ, or the CDζ chain. The domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding.

Second-generation CARs include a co-stimulatory signal in addition to the CD3ζ signal. Coincidental delivery of the delivered co-stimulatory signal enhances cytokine secretion and antitumor activity induced by CAR-transduced T-cells. The co-stimulatory domain is usually be membrane proximal relative to the CD3ζ domain. Third-generation CARs include a tripartite signaling domain, comprising for example a CD28, CD3ζ, OX40 or 4-1BB signaling region. In fourth generation, or “armored car” CAR T-cells are further gene modified to express or block molecules and/or receptors to enhance immune activity.

CAR variants include split CARs wherein the extracellular portion, the ABR sequence and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Trans/Med (2013);5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immuno/Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol(2010) 956304; the disclosures of which are incorporated herein by reference in their entirety.

CAR variants also include bispecific or tandem CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. Tandem CARs (TanCAR) mediate bispecific activation of T cells through the engagement of two chimeric receptors designed to deliver stimulatory or costimulatory signals in response to an independent engagement of two different tumor associated antigens. iCARs use the dual antigen targeting to shut down the activation of an active CAR through the engagement of a second suppressive receptor equipped with inhibitory signaling domains

The dual recognition of different epitopes by two CARs diversely designed to either deliver killing through ζ-chain or costimulatory signals, e.g. through CD28, allows a more selective activation of the reprogrammed T cells by restricting Tandem CARs' activity to cancer cells expressing simultaneously two antigens rather than one. The potency of delivered signals in engineered T cells will remain below threshold of activation and thus ineffective in absence of the engagement of a costimulatory receptor. The combinatorial antigen recognition enhances selective tumor eradication and protects normal tissues expressing only one antigen from unwanted reactions.

Inhibitory CARs (iCARs) are designed to regulate CAR-T cells' activity through activation of inhibitory receptors' signaling modules. This approach combines the activity of two CARs, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility.

An ABR sequence can be formatted as a “chimeric bispecific binding member”, where the ABR sequence provides one of the binding specificities in a chimeric polypeptide having dual specificity to two different binding partners (e.g., two different antigens). Non-limiting examples of chimeric bispecific binding members include bispecific antibodies, bispecific conjugated monoclonal antibodies (mab)₂, bispecific antibody fragments (e.g., F(ab)₂, bispecific scFv, bispecific diabodies, single chain bispecific diabodies, etc.), bispecific T cell engagers (BiTE), bispecific conjugated single domain antibodies, microbodies and mutants thereof, and the like. Non-limiting examples of chimeric bispecific binding members also include those chimeric bispecific agents described in Kontermann. MAbs. (2012) 4(2): 182-197; Stamova et al. Antibodies 2012, 1(2), 172-198; Farhadfar et al. Leuk Res. (2016) 49:13-21; Benjamin et al. Ther Adv Hematol. (2016) 7(3):142-56; Kiefer et al. Immunol Rev. (2016) 270(1):178-92; Fan et al. J Hematol Oncol. (2015) 8:130; May et al. Am J Health Syst Pharm. (2016) 73(1):e6-e13; the disclosures of which are incorporated herein by reference in their entirety.

In some instances, a chimeric bispecific binding member may be a bispecific T cell engager (BiTE). A BiTE is generally made by fusing a specific binding member (e.g., a ABR sequence) that binds to a specific peptide-MHC complex, with a second binding domain specific for a T cell molecule such as CD3.

CD3-based bispecific T-cell engager (TCE) is a protein that simultaneously binds through a target antigen on a tumor cell and CD3 on a T-cell to form a TCR-independent artificial immune synapse. Common molecular formats used to create TCE proteins include knob-into-hole format for Fc and light-chain heterodimerization; knob-into-hole format using a common light chain; knob-into-hole triple-chain format; the 2+1 format including a second Fab (Xencor); knob-into-hole triple-chain format; Fab arm exchange; knob-into-hole Cross-MAb 1+1 format; knob into hole CrossMAb; tetravalent scfv Fc fusion; tetravalent HC:LC and scfv fusion; TandAb diabody; tandem scFv, first generation BiTE®format.

In some instances, a chimeric bispecific binding member may be a CAR T cell adapter. As used herein, by “CAR T cell adapter” is meant an expressed bispecific polypeptide that binds the antigen recognition domain of a CAR and redirects the CAR to a second antigen. Generally, a CAR T cell adapter will have two binding regions, one specific for an epitope on the CAR to which it is directed and a second epitope directed to a binding partner which, when bound, transduces the binding signal activating the CAR. Useful CAR T cell adapters include but are not limited to e.g., those described in Kim et al. J Am Chem Soc. (2015) 137(8):2832-5; Ma et al. Proc Natl Acad Sci USA. (2016) 113(4):E450-8 and Cao et al. Angew Chem Int Ed Engl. (2016) 55(26):7520-4; the disclosures of which are incorporated herein by reference in their entirety.

Effector CAR-T cells include autologous or allogeneic immune cells having cytolytic activity against a target cell expressing an antigen of interest. The effector cells have cytolytic activity that does not require recognition through the T cell antigen receptor. In some embodiments, a T cell is engineered to express a CAR. The term “T cells” refers to mammalian immune effector cells that may be characterized by expression of CD3 and/or T cell antigen receptor.

In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Semin Oncol. 42(4):626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344(6184)641-645, “Cancer immunotherapy based on mutation-specific CD4+ T cells in a patient with epithelial cancer”.

In other embodiments, the engineered T cell is allogeneic with respect to the individual that is treated, e.g. see clinical trials NCT03121625; NCT03016377; NCT02476734; NCT02746952; NCT02808442. See for review Graham et al. (2018) Cells. 7(10) E155. In some embodiments an allogeneic engineered T cell is fully HLA matched. However not all patients have a fully matched donor and a cellular product suitable for all patients independent of HLA type provides an alternative. A universal ‘off the shelf’ CAR T cell product provides advantages in uniformity of harvest and manufacture.

Allogeneic T cells can be genetically modified to reduce graft v host disease. For example, the TCRαμ receptor can be knocked out by different gene editing techniques. TCRαμ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-Crel homing endonuclease, etc. See, for example, Eyquem et al. (2017) Nature 543:113-117, in which the TRAC coding sequence is replaced by the CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies CAR-T cells to express an inhibitor of TCRαμ signaling, for example using a truncated form of CD3ζ as a TCR inhibitory molecule.

Allogeneic T cells may be administered in combination with intensification of lymphodepletion to allow CAR-T cells to expand and clear malignant cells prior to host immune recovery, e.g. by administration of Alemtuzumab (monoclonal anti-CD52), purine analogs, etc. The allogeneic T cells may be modified for resistance to Alemtuzumab, and currently in clinical trials. Gene editing has also been used to prevent expression of HLA class I molecules on CAR-T cells, e.g. by deletion of p2-microglobulin, see NCT03166878.

In addition to modifying T cells, induced pluripotent stem (iPS) CAR-T cells can provide a source of allogeneic CAR-T cells. For example, transducing donor T cells with reprogramming factors can restore pluripotency, and are then re-differentiated to T effector cells.

T cells for engineering as described above collected from a subject or a donor may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Expression construct: The ABR construct (e.g. CAR, scFv, antibody, Fc fusion, drug conjugate, etc.) coding sequence may be introduced on an expression vector into a cell to be engineered. The nucleic acid encoding a ABR sequence is inserted into a vector for expression and/or integration. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

For example, a CAR coding sequence may be introduced into the site of the endogenous T cell receptor, e.g. TRAC gene, e.g., using CRISPR technology (see, for example Eyquem et al. (2017) Nature 543:113-117; Ren et a. (2017) Protein & Cell 1-10; Ren et a. (2017) Oncotarget 8(10):17002-17011). CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating virus, such as adenovirus and adenovirus-associated virus (AAV), has also been reported. Recent discoveries of smaller Cas proteins have enabled and enhanced the combination of this technology with vectors that have gained increasing success for their safety profile and efficiency, such as AAV vectors.

Expression vectors may contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Nucleic acids are “operably linked” when placed into a functional relationship with another nucleic acid sequence. For example, DNA for a signal sequence is operably linked to DNA for a polypeptide if it is expressed as a preprotein that signals the secretion of the polypeptide; a promoter or enhancer is operably linked to a coding sequence if it affects the transcription of the sequence; and a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Expression vectors will contain a promoter that is recognized by the host organism and is operably linked to the ABR coding sequence. Promoters are untranslated sequences located upstream (5′) to the start codon of a structural gene (generally within about 100 to 1000 bp) that control the transcription and translation of particular nucleic acid sequences to which they are operably linked. Such promoters typically fall into two classes, inducible and constitutive. Inducible promoters are promoters that initiate increased levels of transcription from DNA under their control in response to a change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus LTR (such as murine stem cell virus), hepatitis-B virus and Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes may be increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning or expressing a ABR sequence are the prokaryotic and yeast, or other eukaryotic cells described above. Examples of useful mammalian host cell lines are mouse L cells (L-M[TK-], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse Sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including T cells, stem cells, etc. can be transfected with the above-described expression vectors for ABR construct expression. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The effect of treatment can be prophylactic in terms of completely or partially preventing infection. Those in need of treatment include those already affected (e.g., those with infection, cancer, etc.) as well as those in which prevention is desired (e.g., those with increased susceptibility to infection, those with an increased likelihood of infection, those suspected of having infection, those suspected of harboring an infection, etc.).

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of a disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent, e.g. an infusion of engineered T cells, and antibody construct, etc., sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A therapeutic treatment may be one in which the subject is infected prior to administration and a prophylactic treatment is one in which the subject is not infected prior to administration. In some embodiments, the subject has an increased likelihood of becoming infected or is suspected of being infected prior to treatment. In some embodiments, the subject is suspected of having an increased likelihood of becoming infected.

As used herein, the term “infection” refers to any state in at least one cell of an organism (i.e., a subject) is infected by a pathogen. A pathogen may be an intracellular pathogen, e.g. certain bacteria, protozoans, and viruses.

Viruses include those that infect, e.g. farm animals including horses, cattle, sheep, pigs, chickens, turkeys, etc., domestic animals including dogs and cats; and viruses that infect humans. In some embodiments a is an RNA virus. An RNA virus is a virus that has RNA (ribonucleic acid) as its genetic material. This nucleic acid is usually single-stranded RNA (ssRNA) but may be double-stranded RNA (dsRNA). Human diseases caused by RNA viruses include AIDS, Ebola hemorrhoragic fever, SARS, influenza, hepatitis C, West Nile fever, polio, and measles.

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

### Polypeptide and Polynucleotide Compositions

Polypeptide constructs and compositions are provided, which comprise a ABR sequence, optionally linked to an effector polypeptide, which effector polypeptide may include, without limitation, chimeric antigen receptors; Fc sequences, toxins; and fragments and derivatives thereof, which polypeptides may be referred to as a ABR construct.

CDR sequences referred to herein have sequences as shown in Table 1.

TABLE 1

| | | |
|---|---|---|
| | | Numbering |
| | | (relative to |
| Sequence identifier | AA sequence | variable region) |

CDR sequences for human HLA-A2 specific library.
Underlined (red) residues are peptidec ontacts

| | | |
|---|---|---|
| SEQ ID NO: 1 (H-CDR1) | TYQMS | residues 31-35 |
| SEQ ID NO: 2 (H-CDR2) | GIVSSGGSTAYADSVKG | residues 50-66 |
| SEQ ID NO: 3 (H-CDR3) | ELLPYYGMDV | residues 99-108 |
| SEQ ID NO: 4 (L-CDR1) | TGTSRDVGGYNYVS | residues 23-36 |
| SEQ ID NO: 5 (L-CDR2) | DVIERSS | residues 52-58 |
| SEQ ID NO: 6 (L-CDR3) | WSFAGSYYV | residues 91-99 |

Randomized CDR sequences for HLA-A2 specific library.

| | |
|---|---|
| SEQ ID NO: 7 (H-CDR3) | ELLPXYGMDY |
| SEQ ID NO: 8 (L-CDR1) | TGTXRDVGGXNYVS |
| SEQ ID NO: 9 (L-CDR3) | WSXAXXYXV |

CDR Sequences selected for HLA-A2/NY-ESO1 N1

| | |
|---|---|
| SEQ ID NO: 10 (H-CDR3) | ELLPYYGMDY |
| SEQ ID NO: 11 (L-CDR1) | TGTNRDVGGYNYVS |
| SEQ ID NO: 12 (L-CDR3) | WSIAQTYGV |

CDR Sequences selected for HLA-A2/NY-ESO1 N4

| | |
|---|---|
| SEQ ID NO: 10 (H-CDR3) | ELLPYYGMDY |
| SEQ ID NO: 4 (L-CDR1) | TGTSRDVGGYNYVS |
| SEQ ID NO: 15 (L-CDR3) | WSAANVYGV |

CDR Sequences selected for HLA-A2/NY-ESO1 N5

| | |
|---|---|
| SEQ ID NO: 16 (H-CDR3) | ELLPEYGMDY |
| SEQ ID NO: 17 (L-CDR1) | TGTLRDVGGYNYVS |
| SEQ ID NO: 18 (L-CDR3) | WSAAMAYGV |

CDR sequences selected for HLA-A2/MART1 MA2

| | |
|---|---|
| SEQ ID NO: 19 (H-CDR3) | ELLPGYGMDY |
| SEQ ID NO: 20 (L-CDR1) | TGTDRDVGGQNYVS |
| SEQ ID NO: 21 (L-CDR3) | WSPAWPYAV |

CDR sequences selected for HLA-A2/MART1 MA5

| | |
|---|---|
| SEQ ID NO: 22 (H-CDR3) | ELLPSYGMDY |
| SEQ ID NO: 23 (L-CDR1) | TGTDRDVGGYNYVS |
| SEQ ID NO: 24 (L-CDR3) | WSPAWPYSV |

CDR sequences selected for HLA-A2/gp100 gp1

| | |
|---|---|
| SEQ ID NO: 10 (H-CDR3) | ELLPYYGMDY |
| SEQ ID NO: 26 (L-CDR1) | TGTDRDVGGKNYVS |
| SEQ ID NO: 27 (L-CDR3) | WSKAQIYWV |

CDR sequences selected for HLA-A2/gp100 gp1

| | |
|---|---|
| SEQ ID NO: 10 (H-CDR3) | ELLPYYGMDY |
| SEQ ID NO: 29 (L-CDR1) | TGTLRDVGGLNYVS |
| SEQ ID NO: 30 (L-CDR3) | WSWAPWYYV |

CDR sequences selected for HLA-A2/KRAS R1

| | |
|---|---|
| SEQ ID NO: 19 (H-CDR3) | ELLPGYGMDY |
| SEQ ID NO: 32 (L-CDR1) | TGTRRDVGGSNYVS |
| SEQ ID NO: 33 (L-CDR3) | WSMADLYTV |

CDR sequences selected for HLA-A2/KRAS R2

| | |
|---|---|
| SEQ ID NO: 19 (H-CDR3) | ELLPGYGMDY |
| SEQ ID NO: 35 (L-CDR1) | TGTMRDVGGHNYVS |
| SEQ ID NO: 36 (L-CDR3) | WSLADLYEV |

CDR sequences for mouse H2-K^{b} specific library.
Underlined residues are peptide contacts.

| | | |
|---|---|---|
| SEQ ID NO: 37 (H-CDR1) | DYNMD | residues 31-35 |
| SEQ ID NO: 38 (H-CDR2) | DINPNNGGTIYNQKFKG | residues 50-65 |
| SEQ ID NO: 39 (H-CDR3) | KPYYGNFAWFAY | residues 95-102 |
| SEQ ID NO: 40 (L-CDR1) | KASEDIYNRLA | residues 24-34 |
| SEQ ID NO: 41 (L-CDR2) | GATSLET | residues 50-56 |
| SEQ ID NO: 42 (L-CDR3) | QQYWSTPLT | residues 89-97 |

Randomized CDR sequences for H2-K^{b} specific library

| | |
|---|---|
| SEQ ID NO: 43 (H-CDR1) | DYXMD |
| SEQ ID NO: 44 (H-CDR2) | XINPNNGGTXYNQKFKG |
| SEQ ID NO: 45 (H-CDR3) | KPYXGXXAWFAY |
| SEQ ID NO: 46 (L-CDR3) | QQYXXXPLT |

CDR sequences selected for TRP2/H-2K^{b} mTr2

| | |
|---|---|
| SEQ ID NO: 47 (H-CDR1) | DYEMD |
| SEQ ID NO: 48 (H-CDR2) | SINPNNGGTMYNQKFKG |
| SEQ ID NO: 49 (H-CDR3) | KPYYGMNAWFAY |
| SEQ ID NO: 50 (L-CDR3) | QQYYALPLT |

CDR sequences selected for TRP2/H-2K^{b} mTr12

| | |
|---|---|
| SEQ ID NO: 51 (H-CDR1) | DYDMD |
| SEQ ID NO: 52 (H-CDR2) | SINPNNGGTIYNQKFKG |
| SEQ ID NO: 53 (H-CDR3) | KPYRGFHAWFAY |
| SEQ ID NO: 54 (L-CDR3) | QQYYGYPLT |

CDR sequences selected for TRP2/H-2K^{b} mTr13

| | |
|---|---|
| SEQ ID NO: 55 (H-CDR1) | DYYMD |
| SEQ ID NO: 56 (H-CDR2) | LINPNNGGTVYNQKFKG |
| SEQ ID NO: 57 (H-CDR3) | KPYTGHPAWFAY |
| SEQ ID NO: 58 (L-CDR3) | QQYYAFPLT |

CDR sequences selected for TRP2/H-2K^{b} mTr19

| | |
|---|---|
| SEQ ID NO: 55 (H-CDR1) | DYYMD |
| SEQ ID NO: 60 (H-CDR2) | TINPNNGGTVYNQKFKG |
| SEQ ID NO: 61 (H-CDR3) | KPYSGHPAWFAY |
| SEQ ID NO: 62 (L-CDR3) | QQYYAFPLT |

In an embodiment, the ABR sequence is covalently linked, e.g. as a single polypeptide fused in frame to an effector polypeptide of a CAR, to an Fc sequence, etc. In an embodiment the ABR sequence is provided as a polypeptide linked to an immunoglobulin effector sequence, for example an Fc sequence.

Also provided are isolated nucleic acids encoding the ABR sequence and constructs thereof, vectors and host cells comprising the nucleic acid, and recombinant techniques for the production of the polypeptide constructs. Nucleic acids of interest encode a polypeptide that is at least about 80% identical to the provided polypeptide sequences, at least about 85%, at least about 90%, at least about 95%, at least about 99%, or identical. Polynucleotide sequences may encode any or all of the provided sequences, or may encode a fusion protein such as an Fc fusion, a CAR.

In some embodiments, a vector comprising a coding sequence that encodes ABR sequence or ABR construct is provided, where the coding sequence is operably linked to a promoter active in the desired cell; or is provided in a vector suitable for genomic insertion, e.g., by CRISPR. Various vectors are known in the art and can be used for this purpose, e.g., viral vectors, plasmid vectors, minicircle vectors, which vectors can be integrated into the target cell genome, or can be episomally maintained.

Polypeptide compositions may be prepared as aerosols, injectables, either as liquid solutions or suspensions; solid forms suitable for solution in, or suspension in, liquid vehicles prior to injection can also be prepared. Proteins can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In another embodiment of the invention, an article of manufacture containing an isolated polypeptide or polynucleotide is provided. The article of manufacture comprises a container and a label. Suitable containers include, for example, bottles, vials, syringes, and test tubes. The containers may be formed from a variety of materials such as glass or plastic. The container holds a polypeptide or polynucleotide composition, which may be a therapeutic composition, e.g. for treatment of cancer, and may have a sterile access port (for example the container may be an intravenous solution bag or a vial having a stopper pierceable by a hypodermic injection needle). A label on or associated with the container may indicate that the composition is used for treating the condition of choice. Further container(s) may be provided with the article of manufacture which may hold, for example, a pharmaceutically-acceptable buffer, such as phosphate-buffered saline, Ringer's solution or dextrose solution. The article of manufacture may further include other materials desirable from a commercial and user standpoint, including other buffers, diluents, filters, needles, syringes, and package inserts with instructions for use.

Engineered cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow drip), intraarterial, intraperitoneal, intrathecal or subcutaneous administration.

### Methods of Treatment

The invention further provides methods for treating cancer, reducing viral infection, etc. The ABR sequence may be conjugated to a drug that reduces cell growth, e.g. chemotherapeutic drug, toxin, etc. Thus, in some embodiments, the invention provides a method of delivering a drug to a cell, comprising administering a drug-ABR complex to a subject. Targeting can be accomplished by coupling (e.g., linking, directly or via a linker molecule, either covalently or non-covalently, so as to form a drug-antibody complex) a drug to an antibody specific for a cancer-associated polypeptide. Methods of coupling a drug to a protein are well known in the art.

The types of cancer that can be treated using the subject methods of the present invention include but are not limited to adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

In some embodiments, a cancer is treated with a specific ABR containing construct, where the antigenic peptide is a cancer-associated peptide.

In one embodiment, constructs comprising ABR specific for NY-ESO1 are utilized in the treatment of cancers in which NY-ESO-1 expression has been reported, e.g. including neuroblastoma, myeloma, metastatic melanoma, synovial sarcoma, bladder cancer, esophageal cancer, hepatocellular cancer, head and neck cancer, non-small cell lung cancer, ovarian cancer, prostate cancer, and breast cancer.

In one embodiment, constructs comprising ABR specific for MART1 are utilized in the treatment of cancers in which MART1 expression has been reported, particularly melanomas, and including metastatic melanomas that may reside as distant sites. Also of interest for the treatment of melanoma are constructs comprising ABR specific for gp100, (also termed Pmel17 for premelanosomal protein), which is a membrane-bound protein that is expressed in melanocytes and pigmented cells in the retina and in most malignant melanomas.

In one embodiment, constructs comprising ABR specific for KRAS are utilized in the treatment of cancers in which KRAS expression has been reported. KRAS is a major driver in lung adenocarcinomas, colorectal cancer, and in pancreatic cancers. It can be widely expressed in a number of other cancers, however, including, for example, epithelial cancers.

### Methods of Screening

Compositions and methods are provided for accurately identifying proteins that specifically bind to a peptide in a given MHC context, where the peptide may be a sequence of from about 8 to about 20 amino acids in length, usually from about 8 to about 18 amino acids, from about 8 to about 16 amino acids, from about 8 to about 14 amino acids, from about 8 to about 12 amino acids, from about 10 to about 14 amino acids, from about 10 to about 12 amino acids.

ABRs that have affinity for an MHC-peptide sequences of interest are identified. In such methods, a structural analysis can be performed on the interaction between an initial ABR that binds to an initial MHC protein of interest, in combination with an initial peptide. The structural analysis is used to pinpoint the specific CDR residues that contact the peptide portion of the antigen. An library of polynucleotide sequences encoding ABRs is generated by randomization of the CDR residues thus identified. The number of contact residues to be randomized is determined by the ABR structure, and may vary from about 7 to about 10 residues.

The library of ABRs may be formatted as single chain variable regions (scFv) operably linked to an expression vector. The ABR coding sequence may be fused to a domain that allows the polypeptide to be tethered to a cell surface, including without limitation yeast Aga2, or is a transmembrane domain that allows display on a cell surface. In some embodiments the library comprises a diversity of at least 10 different sequences, at least 5×10⁷ different sequences, at least 10⁸ different sequences, and may comprise at least 5×10⁸ different sequences. For screening, the library is introduced into a suitable host cell that expresses the encoded polypeptide, which host cells include, without limitation, yeast cells.

The library can be provided in the form of a polynucleotide, e.g. a coding sequence operably linked to an expression vector; which is introduced by transfection, electroporation, etc. into a suitable host cell. Eukaryotic cells are preferred as a host, and may be any convenient host cell that can be transfected and selected for expression of a protein on the cell surface. Yeast cells are a convenient host, although are not required for practice of the methods. Once introduced in the host cells, expression of the library is induced and the cells maintained for a period of time sufficient to provide cell surface display of the polypeptides of the library.

A soluble MHC-peptide complex, optionally a multimerized complex to enhance binding, is used to select for host cells expressing an ABR that specifically binds to the complex. The MHC component is generally the initial MHC protein, but the peptide component may be distinct from the initial peptide. Iterative rounds of selection are performed, i.e. the cells that are selected in the first round provide the starting population for the second round, etc. until the selected population has a signal above background, usually at least three and more usually at least four rounds of selection are performed. Polynucleotides encoding the final selected ABR from the library can be reformatted into various configurations.

Selection for a protein that binds to the MHC-peptide of interest is performed by combining a pMHC complex with the population of host cells expressing the library. See Overall S. A. et al., and Sgourakis N. G, Nature Communications (2020) and WO 2020/010261 each herein specifically incorporated by reference. The multimerized MHC-peptide for selection is a soluble protein comprising the binding domains of an MHC of interest complexed with a peptide, and can be synthesized by any convenient method. The MHC may be a single chain, or a multimer, dendrimer, etc., and can comprise a detectable label, e.g. a fluorophore, mass label, etc., or can be bound to a particle, e.g. a paramagnetic particle. Selection of cells bound to the MHC-peptide can be performed by flow cytometry, magnetic selection, and the like as known in the art.

Rounds of selection are performed until the selected population has a signal above background, usually at least three and more usually at least four rounds of selection are performed. In some embodiments, initial rounds of selection, e.g. until there is a signal above background, are performed with an MHC coupled to a magnetic reagent, such as a superparamagnetic microparticle, which may be referred to as “magnetized”. Alternatively, one may also use second stage antibodies that recognize species-specific epitopes of the MHC, e.g. anti-mouse Ig, anti-rat Ig, etc. Indirect coupling methods allow the use of a single magnetically coupled entity, e.g. antibody, avidin, etc., with a variety of separation antibodies.

Alternatively, e.g. for final rounds of selection, the MHC is multimerized to a reagent having a detectable label, e.g. for flow cytometry, mass cytometry, etc. For example, FACS sorting can be used to increase the concentration of the cells of having a peptide ligand binding to the TCR. Techniques include fluorescence activated cell sorters, which can have varying degrees of sophistication, such as multiple color channels, low angle and obtuse light scattering detecting channels, impedance channels, etc.

After a final round of selection, polynucleotides are isolated from the selected host cells, and the sequence determined, usually by high throughput sequencing. The desired affinity may be at less than about 10⁻⁷ kd less than about 10⁻⁸, less than about 10⁻¹, less than about 10⁻¹, less than about 10-1.

The peptide sequence results and database search results may be provided in a variety of media to facilitate their use. “Media” refers to a manufacture that contains the expression repertoire information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression repertoire.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Structure-guided rational approach allows for the design of TCR mimic antibody libraries that bind a target peptide displayed on MHCs. Yeast display libraries expressing single chain variable fragments (scFvs) were constructed with randomized amino acids (excluding cysteine) in the antigen binding CDR domains. Sequences binding to specific peptide-MHC complexes (pMHC) were selected via yeast display screening against biotinylated pMHCs. Finally, the binding affinities, binding specificity, and functional activities of TCRm antibodies such as ADCC activity or cytotoxic activity, were assessed.

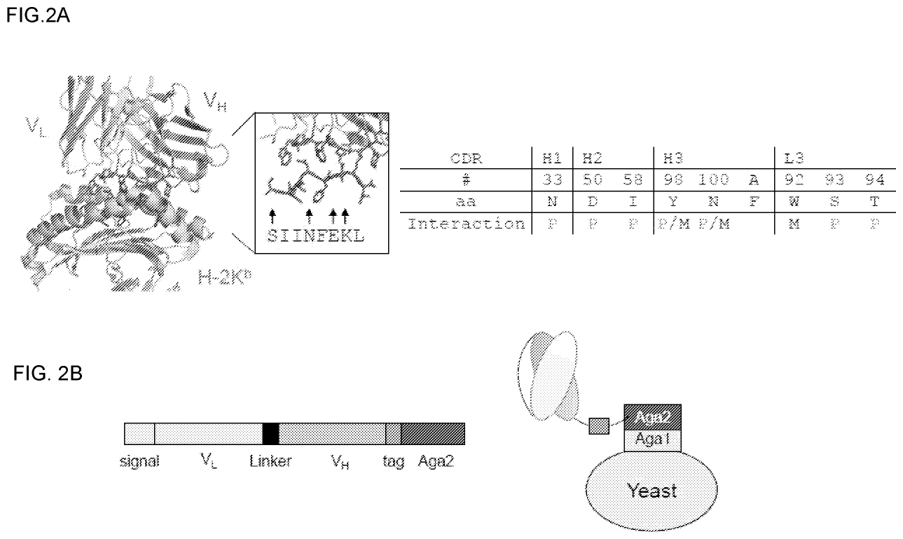

Shown in FIG. 2, (a) the amino acid residues of TCR like antibody for OVA/H-2K^{b} (PDB; 3cvh) in close contact (4 Å) to a peptide displayed on H-2K^{b} were calculated and picked up by Collaborative Computational Project 4 (CCP4) and PyMOL. The variable light chain domain (V_{L}) was colored cyan, the variable heavy chain domain (V_{H}) was colored purple, and MHC was colored green. Contact residues of antibody were colored magenta, and the OVA peptide was colored red. By introducing randomized amino acids excluding Cys into the selected 9 residues, a mouse TCRm scFv yeast library at the diversity of 5×10⁸ was created. (b) Schematic representation of the construct for the TCR mimic scFv yeast display library.

Nine amino acid residues of TCR like antibody for OVA/H-2K^{b} in close contact (4 Å) to a peptide displayed on H-2K^{b} were calculated and picked up by Collaborative Computational Project 4 (CCP4) and PyMOL.

The randomized scFv library construct was divided into six oligonucleotide fragments for gene synthesis (below). The first fragment was amplified by PCR. Three of six oligonucleotides encoding the variable region residues were randomly synthesized using Trimer Codon Mix 2 Sense (Gene Link). The remaining two oligonucleotides were synthesized commercially (Integrated DNA technologies). The five synthesized oligonucleotides were mixed and annealed in the buffer (10 mM Tris-HCl, 1 mM EDTA, 100 mM NaCl, pH 8). Then, an overlap PCR was performed using the first fragment and the annealed fragment. Finally, yeast libraries were created by electroporation of competent EBY-100 cells via homologous recombination of linearized pYAL vector and the resulting PCR product essentially as described previously (Chao (2006) Nat Protoc. 2006; 1(2):755-68. doi: 10.1038/nprot.2006.94).

### The Primers for 1^{st} Fragment

Primer 1
(SEQ ID NO: 63)
5′-ATGCAGTTACTTCGCTGTTTTTCAATATTTTCTGTTATTGCTAGCG-3′
Primer 2
(SEQ ID NO: 64)
5′-GCTGACAGTAATAAGTGGCTACATCCTCCGTC-3′;
Synthesized fragment A
(SEQ ID NO: 65)
GACGGAGGATGTAGCCACTTATTACTGTCAGCAATACnnnnnnnnnCCGCTGACTTTCGGGG
CTGGGACAAAGCTAGAGC;
Synthesized fragment B
(SEQ ID NO: 66)
CGCTGACTTTCGGGGCTGGGACAAAGCTAGAGCTTAAAGGCGGAGGTGGATCAGGTGGA
AGTGGGGGGGGAGGGGGGGGAAGCGAAGTATTATTACAACAGTCAGGCCCCGAATTAGT
GAAACCAGGAGCTTCTGTCAAGATCCCGTGCAAGGCAAGCGGGTATACTTTTACCG;
Synthesized fragment C
(SEQ ID NO: 67)
CCGTGCAAGGCAAGCGGGTATACTTTTACCGATTATnnnATGGATTGGGTTAAACAAAGCC
ATGGCAAATCATTGGAATGGATAGGAnnnATTAATCCCAATAACGGAGGCACTnnnTACAAT
CAAAAGTTCAAAGGTAAGGCTACC;
Synthesized fragment D
(SEQ ID NO: 68)
CAATCAAAAGTTCAAAGGTAAGGCTACCTTGACGGTGGATAAGAGTTCATCAGCCGCATAC
ATGGAGGTGAGGTCCCTTACATCCGAGGACACCGCTGTATACTACTGTGCTAGGAAACCG;
Synthesized fragment E
(SEQ ID NO: 69)
CCGCTGTATACTACTGTGCTAGGAAACCGTATnnnGGGnnnnnnGCATGGTTCGCCT
ACTGGGGTCAGGGGACTCTAGTCACTGTATCAGCGgGATCC;

### Primers for the Final Fragment

Primer1, Primer 3
(SEQ ID NO: 70)
5′-CAGCGTAGTCTGGAACGTCGTATGGGTAGGATCcCGCTGATACA
GTGACTAG-3′;

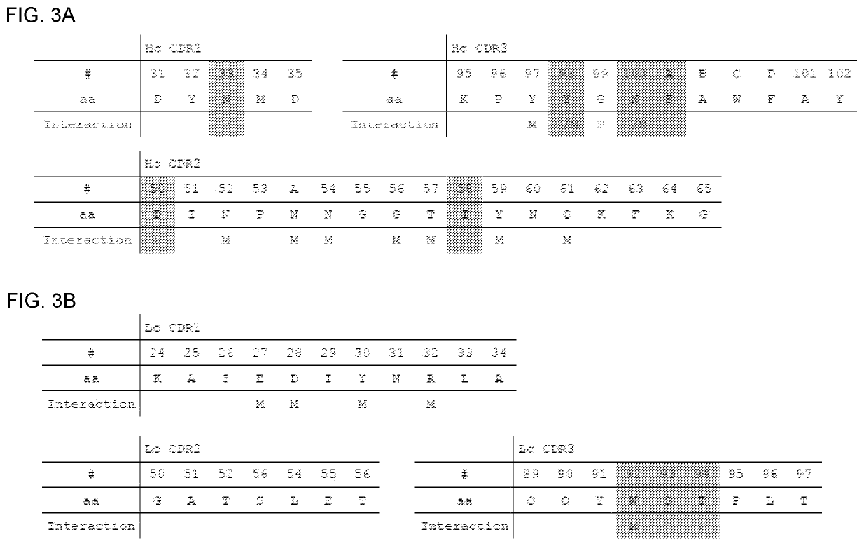

Shown in FIG. 3 are the CDR sequences for mouse TCR mimic Ab; a) heavy chain and b) light chain. Interacting residues of an antibody with the OVA peptide of pMHC were marked as “P” and interacting residues with MHC were marked as “M”. Nine interacting residues on CDRs were selected, and the randomized amino acids (except for Cys) were introduced into the amino acid residues which were highlighted in pink to make a TCR mimic scFv yeast library; (a) heavy chain and (b) light chain.

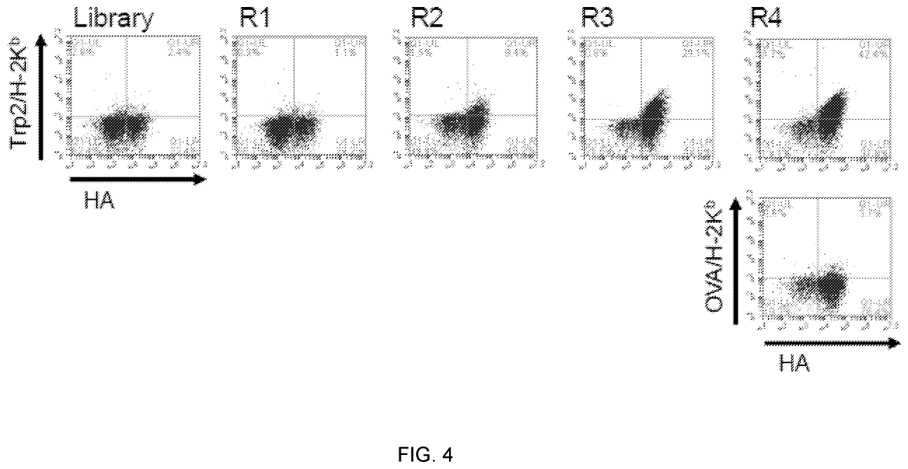

Yeast display selection against mouse Trp2/H-2K^{b} was performed using mouse TCR mimic scFv library. Specific enrichment for binders was observed after 4 rounds of selection. The panels indicate tetramer pMHC staining on the Y axis and HA-tag staining of the scFv on the X-axis, shown in FIG. 4.

Method for Expression and Purification of biotinylated single chain pMHC. The mouse Trp2/H-2K^{b} was constructed as a single chain pMHC (sc pMHC). The Trp2 peptide (SVYDFFVWL (SEQ ID NO:90)), beta2M, H-2K^{b} (without the transmembrane domain), biotinylation tag, and His-tag were linked by (G₄S)_{3 or 4} (SEQ ID NO:89) linkers. The sc pMHC construct was expressed in Expi293 cells, and then purified with Ni-NTA resin. Site-specific biotinylation of biotinylation tag was performed at 4° C. overnight in the presence of excess biotin (>1 mM), BiomixA, BiomixB, and BirA, followed by size exclusion chromatography using the Superdex200 column (GE Healthcare). Biotinylation was confirmed by band-shift analysis based on SDS-PAGE. 1-2 μg of pMHC was boiled prior SDS-PAGE. The pMHC was mixed with or without streptavidin, and then the mixtures were subjected to SDS-PAGE.

Before each round of selection, a small sample of yeast (˜1×10⁶ cells) was stained with tetramer pMHC and an anti-HA tag (6E2 antibody AlexaFluor 488 conjugate, Cell Signaling Technology) for 1 hr on ice to confirm the surface expression of the scFv-Aga2 library. For tetramer pMHC staining, biotinylated Trp2/H-2K^{b} was incubated with streptavidin coupled to AlexaFluor 647. After washing cells with PBS-M (PBS, 0.5% BSA, and 1 mM EDTA) twice, cells were analyzed using Accuri C6 flow cytometer (BD Biosciences). Ovalbumin (OVA)/H-2K^{b} was used as a negative control.

For the first round of selection, approximately 2×10⁹ cells were incubated with 250 μL Streptavidin Microbeads (Miltenyi Biotec) for 1 hr at 4° C. Cells passed through an LS column (Miltenyi Biotec) to remove nonspecifically binding cells. Then, 400 nM of biotinylated pMHC coupled to 250 μL of Streptavidin Microbeads was incubated with cells for 3-4 hr at 4° C. The pMHC-binding yeasts were collected via an LS column, washed with PBS-M (PBS, 0.5% BSA, 2.5 mM EDTA), and then re-cultured in SD-CAA at 30° C. for 1-2 days. Yeasts were induced by SG-CAA at 20° C. for 2-3 days. Second and third rounds of selection was performed in the same manner, but with reduced amount of Streptavidin Microbeads (50 μL). At fourth round of selection, 40 nM of biotinylated pMHC was used after negative screening with OVA/H-2K^{b} for 1 hr.

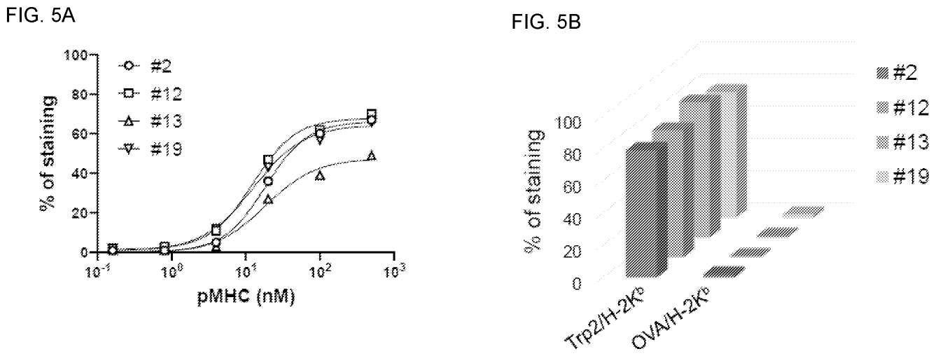

FIG. 5 shows the binding characteristics of individual yeast clones expressing scFv for Trp2/H-2K^{b}. The top 4 candidates were selected via monomer and tetramer pMHC staining. (a) The individual yeast clones bound to monomer Trp2/H-2K^{b} in a dose-dependent manner. (b) Yeast clones specifically bound to 100 nM of Trp2/H-2K^{b}, compared with 1,000 nM of OVA/H-2K^{b}. Data are representative of two independent experiments.

A small amount of yeast clones expressing TCRm scFv were incubated with 10-fold serially diluted Trp2/H-2K^{b} for 1 hr on ice. After washing cells with PBS-M, cells were stained with streptavidin coupled to AlexaFluor 647 for 15 min on ice and washed. Cells were analyzed by Accuri C6 flow cytometer. To assess binding specificity, cells were stained with 100 nM of tetramer Trp2/H-2K^{b} or 1,000 nM of tetramer OVA/H-2K^{b} for 1 hr on ice. After washing cells with PBS-M, cells were analyzed by Accuri C6 flow cytometer. Assays were performed in biological duplicates.

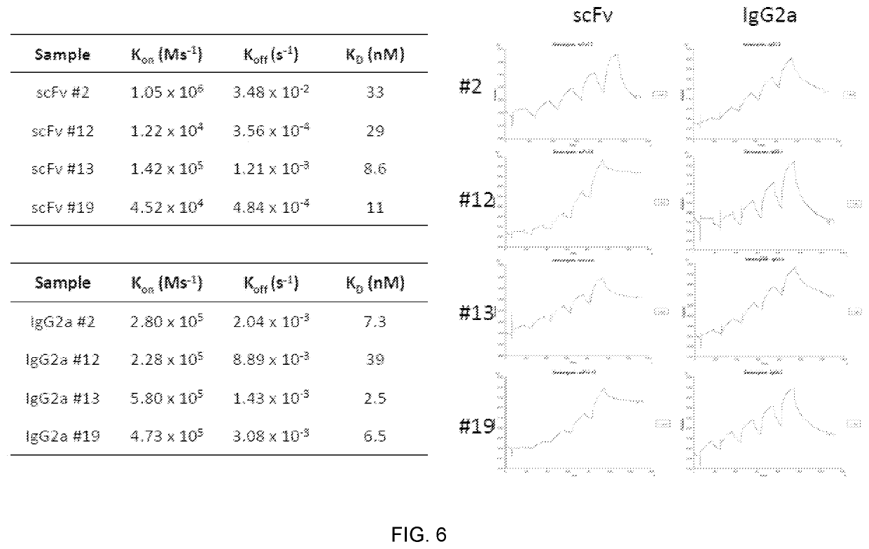

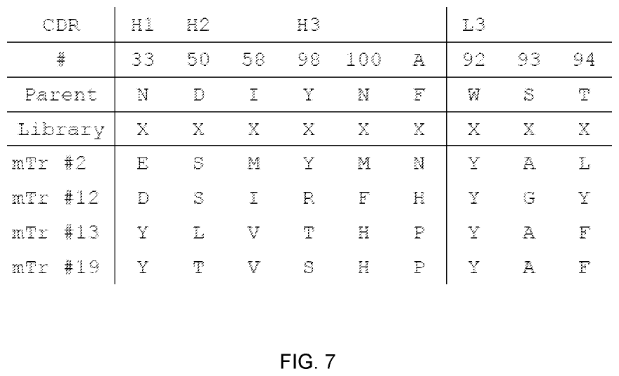

Surface plasmon resonance of mouse TCRm Abs binding to Trp2/H-2K^{b} (FIG. 6). The binding affinities of mouse TCR mimic scFvs and IgGs were measured by biacoreT100 at room temperature. BIAcore T100 (GE Healthcare) was used to measure the K_{D} by single cycle kinetics method. The pMHC was immobilized on a SA chip at 10-100 response unit (RU). Purified samples (scFv and IgG) were captured by injection of varying conc (0.008-200 nM) with HBS-EP for 120 see at a flow of 30 μL/min, then dissociation measured for 300 see with buffer flow. The signal of reference cells was subtracted from the measurements. Data analysis was performed using BIAcore T100 evaluation software. All the sample is representative of at least two independent experiments.

Method for Expression and Purification of scFv and IgG. Candidates of scFv and mouse IgG2a were expressed in Expi293 cells, and then purified with Ni-NTA resin or proteinG Sepharose FF resin.

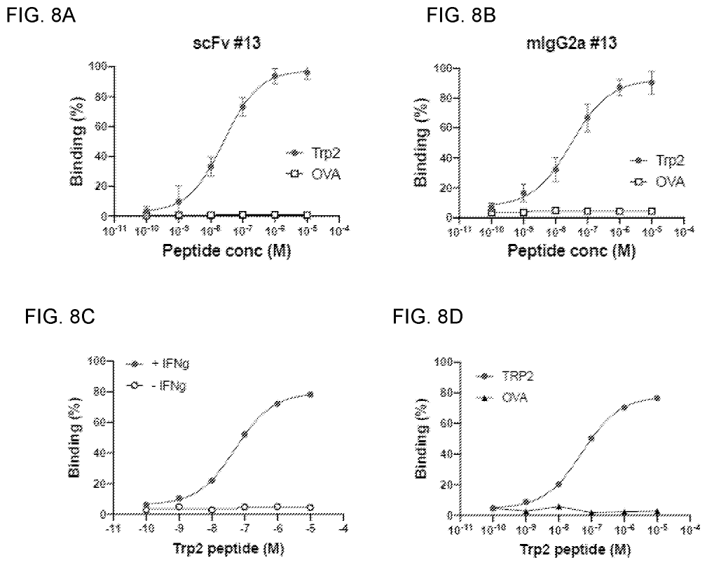

As shown in FIG. 8, B16F10 murine melanoma cells were incubated with 100 U/mL of mouse IFN-gamma overnight. Ten-fold serial dilutions of Trp2 peptide or OVA peptide were pulsed to EL4 cells or B16F10 cells for 1 hr at 37° C., then washed with PBS once. One μg/mL of scFv #13 or IgG #13 was mixed with cells for 1 hr at 37° C. After washing cells with PBS, 6-His tag antibody FITC conjugated (Bethyl) for detection of scFv or proteinG AlexaFluor 488 conjugated for detection of IgG was incubated with cells for 1 hr. After washing cells with PBS-M several times, cells were analyzed by Accuri C6 flow cytometer (BD Biosciences). Assays were performed in biological and technical triplicates.

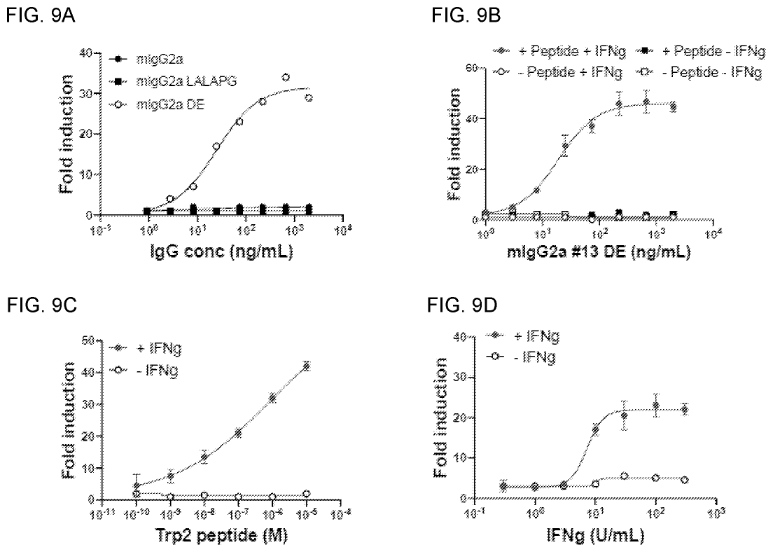

B16F10 cells were induced by 100 U/mL of mouse IFN-gamma overnight. 5,000 cells mixed with 10 μM of Trp2 peptide in assay buffer (Promega) were plated in 96-well white flat-bottom plates for 1 hr at 37° C., and then cells were incubated together with 3-fold serial dilutions of samples. The mFcγRIV effector cells (Promega) were resuspended in assay buffer and then added to assay plates at a concentration of 1×10⁵ cells per well. After incubation of cells at an Effector:Target (E:T) ratio of 20:1 for 6 hr at 37° C., Bio-Glo Luciferase Assay Reagent (Promega) was added, and luminescence was measured by SpectraMax Paradigm microplate reader (Molecular Devices), data shown in FIG. 9.

For the peptide titration, 10-fold dilutions of Trp2 peptide and 1 ug/mL of mIgG2a DE were used. For the IFN-gamma titration, 3-fold dilutions of IFN-gamma were incubated with B16F10 cells overnight before ADCC assay, and 10 μM of Trp2 peptide and 1 μg/mL of mIgG2a DE were used. Fold induction was calculated as ADCC activity according to the manufacture's instrument (Promega). Assays were performed in biological duplicates or triplicates.

The L238A, L239A, and P333G (LALAPG) mutations in the mIgG2a heavy chain were introduced for functionally silent Fc mutant. The S243D and 1336E (DE) mutations in the mIgG2a heavy chain were introduced for functionally enhanced Fc mutant. Both antibodies with engineered Fc mutants were expressed in Expi293 cells, followed by purification of proteinG Sepharose FF resin.

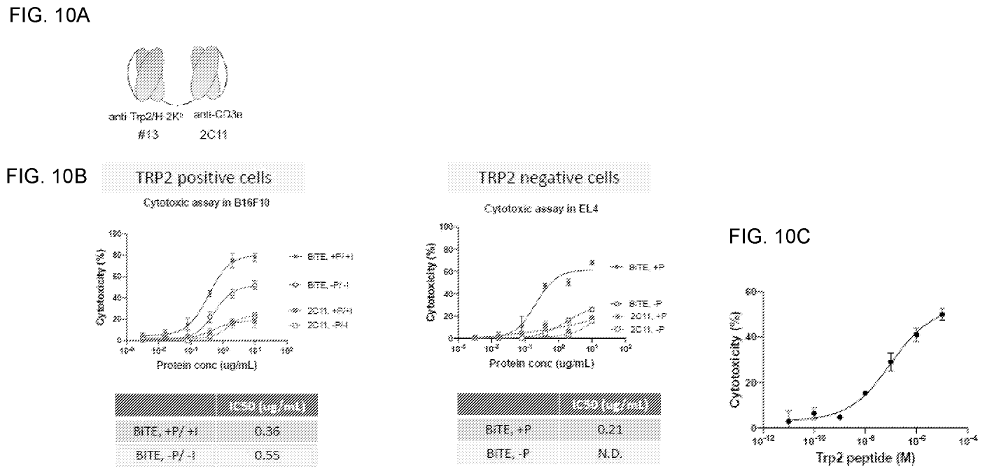

Shown in FIG. 10, Spleen and lymph node were isolated from BL6 mice. Mouse T lymphoblasts were activated in RPMI complete medium including 100 IU/mL mlL2 on plates coupled with 2.5 μg/mL of 2C11 anti-mouse CD3 antibody (eBioscience) and 5 μg/mL of anti-mouse CD28 antibody (BioXCell). After 2-days cultivation, mouse T lymphoblasts were passaged and cultured in RPMI complete medium including 100 IU/mL of mlL2 every 2-3 days until day 15. One day before starting cytotoxic assays, mouse T lymphoblasts were harvested and resuspended in RPMI complete medium without mlL2, and then used as effector cells.

One hundred U/mL of mouse IFN-gamma (R&D systems) was incubated with B16F10 cells overnight. Cells were labeled with 2 μM of CFSE (Invitrogen) for 15 min. After washing cells with PBS, 1 μM of Trp2 peptide was pulsed to 2×10⁵ cells/mL for 1 hr at 37° C. Five-fold serial dilutions of BiTE were added to 10⁴ cells. Then, mouse T cell lymphoblasts were incubated at a 1:1 Effector:Target (E:T) ratio. After incubation for 48 hrs, cells were resuspended in Propidium Iodide (ThermoFisher). CFSE and PI double positive cell populations were gated and analyzed by CytoFLEX Flow Cytometer (Bechman). For the peptide titration, 10-fold dilutions of Trp2 peptide and 0.5 μg/mL BiTE were used. Percentage of specific lysis was subsequently calculated as follows; cytotoxicity (%)=[1-(BiTE-treated units/no BiTE control units)]*100. Assays were performed in biological duplicates or triplicates.

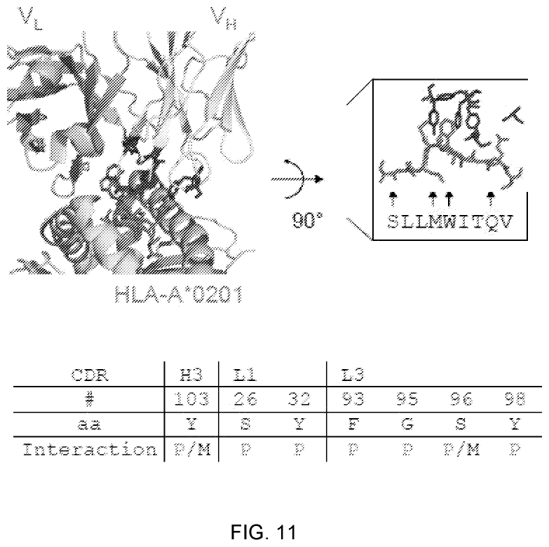

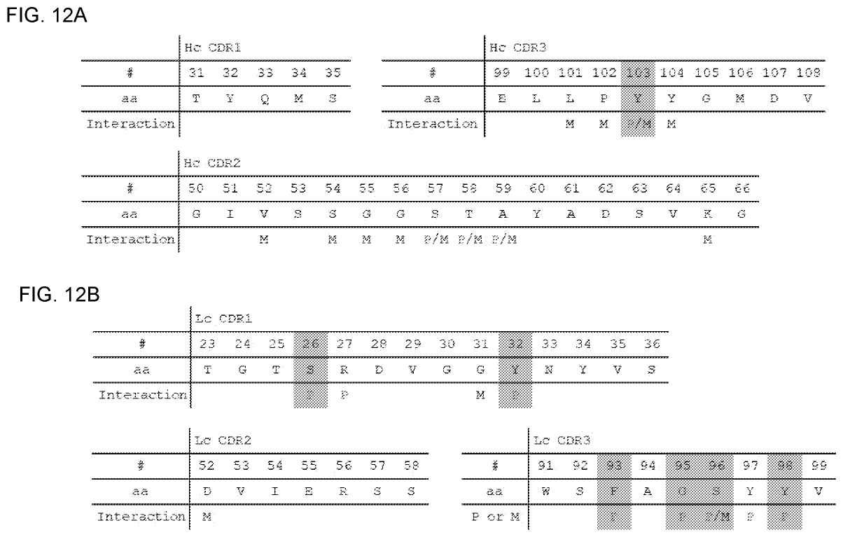

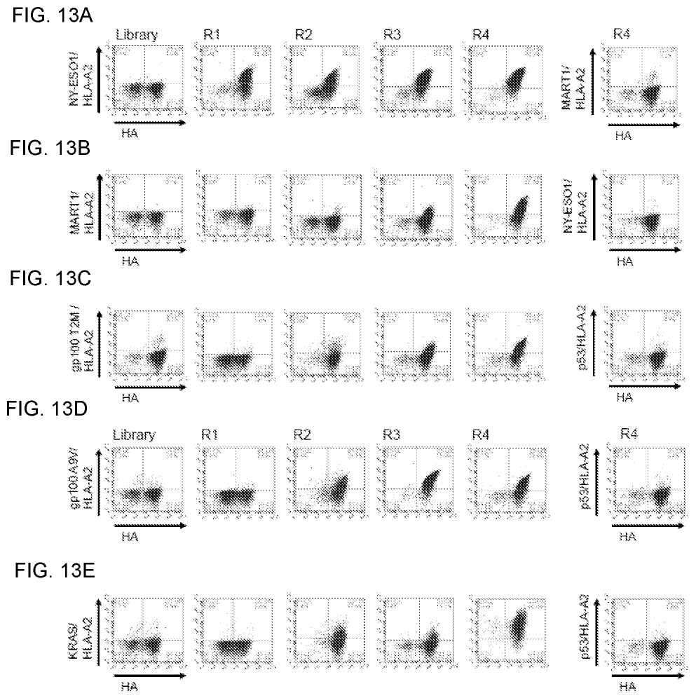

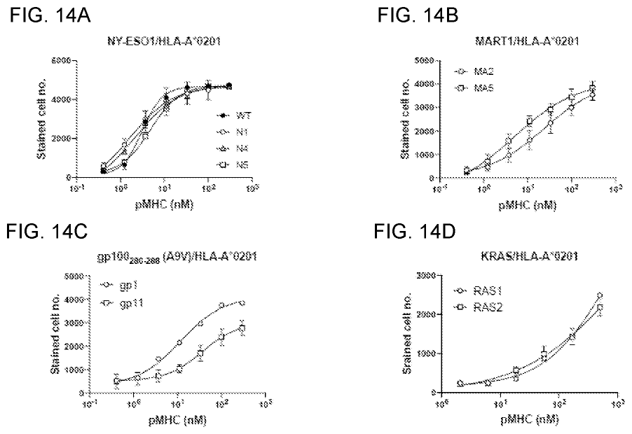

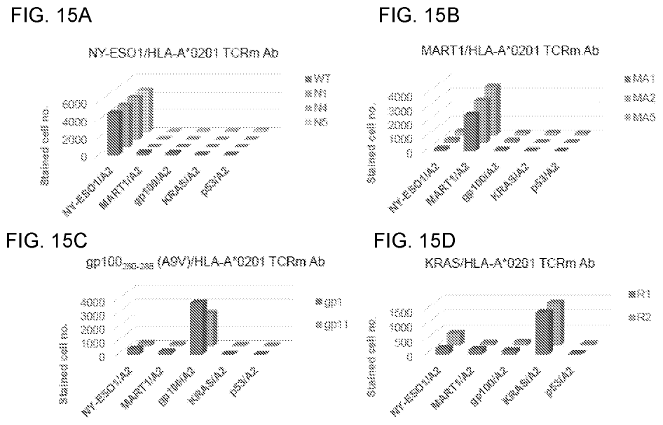

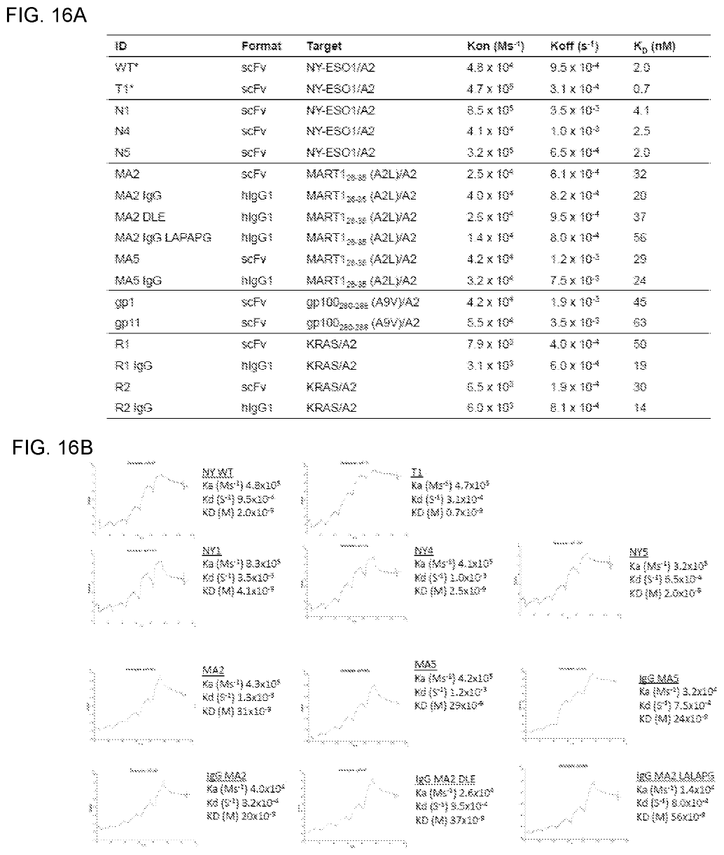

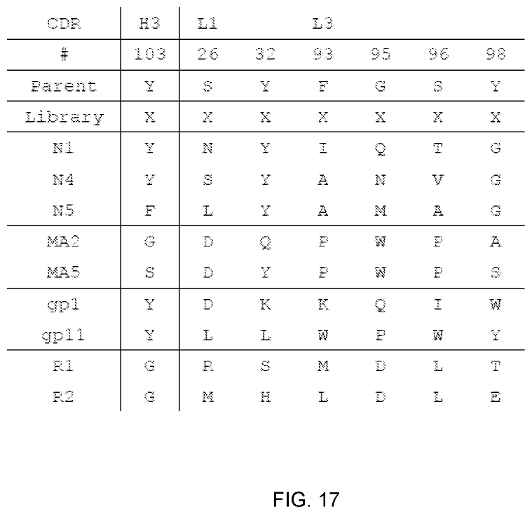

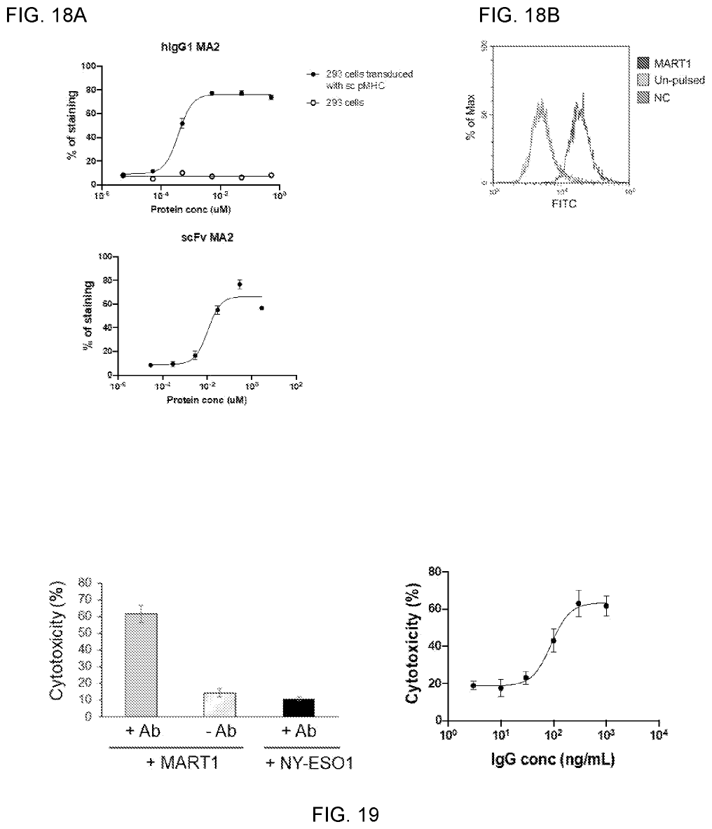

Design and construction of human TCR mimic scFv Library (FIG. 11). Seven amino acid residues of TCR like antibody for NY-ESO1/HLA-A2 in close contact (4 Å) to a peptide displayed on HLA-A*0201 were calculated and picked up by Collaborative Computational Project 4 (CCP4) and PyMOL.

The randomized scFv library construct was divided into 6 oligonucleotide fragments for gene synthesis. The first fragment was amplified by PCR. Three of six oligonucleotides encoding the variable region residues were randomly synthesized using Trimer Codon Mix 2 Sense (Gene Link). The remaining two oligonucleotides were synthesized commercially (Integrated DNA technologies). The five synthesized oligonucleotides were mixed and annealed in the buffer (10 mM Tris-HCl, 1 mM EDTA, 100 mM NaCl, pH 8). Then, an overlap PCR was performed using the first fragment and annealed fragment. Finally, yeast libraries were created by electroporation of competent EBY-100 cells via homologous recombination of linearized pYAL vector and the resulting PCR product essentially as described previously (reference; 2006 Chao).

### Primers for 1^{st} Fragment

Primer 4
(SEQ ID NO: 63)
5′-ATGCAGTTACTTCGCTGTTTTTCAATATTTTCTGTTATTGCTAG
CG-3′;
Primer 5;
(SEQ ID NO: 72)
5′-CAACAGCTCCCCCGCACAGTAGTAAACGGCAGTATCCTC-3′
Synthesized fragment F
(SEQ ID NO: 73)
CGAGGATACTGCCGTTTACTACTGTGCGGGGGAGCTGTTGCCCnnnTAT
GGTATGGACGTTTGGGGACAAGGCACGACGGTCACGGTGTCATC
Synthesized fragment G
(SEQ ID NO: 74)
GGACGTTTGGGGACAAGGCACGACGGTCACGGTGTCATCTGGAGGAGGT
GGTTCCGGAGGTGGGGGTTCCGGGGGGGGAGGTAGCCAATCAGAGCTGA
CCCAGCCTAGAAGTGTCAGTGGTTCACCCGGTCAATCCGTAACTATTTC
TTGCACAGG;
Synthesized fragment H
(SEQ ID NO: 75)
CAGTGGTTCACCCGGTCAATCCGTAACTATTTCTTGCACAGGCACCnnn
AGGGACGTGGGGGGAnnnAATTACGTGTCCTGGTACCAACAACATCCGG
GAAAAGCCCCCAAATTG;
Synthesized fragment I
(SEQ ID NO: 76)
GTGTCCTGGTACCAACAACATCCGGGAAAAGCCCCCAAATTGATAATCC
ATGACGTTATTGAAAGAAGCTCAGGAGTCCCAGACAGGTTCAGGGGTAG
CAAGAGTGGCAATACCGCAAGCCTGACTATCTCTGGGCTGCAGGCCGAA
GACGAAGCAGACTATTATTGCTGG;
Synthesized fragment J
(SEQ ID NO: 77)
GGGCTGCAGGCCGAAGACGAAGCAGACTATTATTGCTGGAGTnnnGCGn
nnnnnTATnnnGTCTTCGGGACGGGAACGGACGTGACCGTCTTAgGATC
CTACCCATACGACGTTCCAGACTACGCTgg;

### Primers for Fragment 2;

### Primer 4

Primer 6
(SEQ ID NO: 78)
5′-ccAGCGTAGTCTGGAACGTCGTATGGGTAGG-3′;

Method for Expression and Purification of biotinylated single chain pMHC. The human NY-ESO1₁₅₇₋₁₆₅/HLA-A*0201, MART1₂₆₋₃₅ (A2L)/HLA-A*0201, gp100₂₀₉₋₂₁₈ (T2M)/HLA-A*0201 and gp100₂₈₀₋₂₈₈ (A9V)/HLA-A*0201 were produced as single chain pMHC (sc pMHC). The target peptide, beta2M, HLA-A*0201 without the transmembrane domain, biotinylation tag, and His-tag were linked by (G₄S)_{3 or 4} (SEQ ID NO:89) linkers. The sc pMHCs were expressed in Expi293 cells, and then purified with Ni-NTA resin. Site-specific biotinylation of biotinylation tag was performed at 4° C. overnight in the presence of excess biotin (>1 mM), BiomixA, BiomixB, and BirA, followed by size exclusion chromatography using the Superdex200 column. Biotinylation was confirmed by band-shift analysis based on SDS-PAGE. 1-2 μg of biotinylated pMHCs were boiled prior SDS-PAGE. The pMHCs were mixed with or without streptavidin, and then the mixtures were subjected to SDS-PAGE.

### The Sequences of Target Peptides

NY-ESO₁₁₅₇₋₁₆₅;
(SEQ ID NO: 79)
SLLMWITQV
MART1₂₆₋₃₅ (A2L)
(SEQ ID NO: 80)
ELAGIGILTV;
gp100₂₀₉₋₂₁₈ (T2M)
(SEQ ID NO: 81)
IMDQVPFSV;
gp100₂₈₀₋₂₈₈ (A9V)
(SEQ ID NO: 82)
YLEPGPVTV;
KRAS₅₋₁₄;
(SEQ ID NO: 83)
KLVVVGAGGV

Method for refolding and biotinylation of KRAS₅₋₁₄/HLA-A*0201. The human beta2M and HLA-A*0201 without the transmembrane domain were separately cloned into pET28b vector (Novagen) and expressed as inclusion bodies in BL21 (DE3) (Novagen). The inclusion bodies were purified with the ultrasonic sonicator (Branson) and dissolved in 8 M Urea buffer (8 M Urea, 20 mM Tris-HCl pH 8.0, 0.5 mM EDTA, 1 mM DTT). Thirty mg of KRAS₅₋₁₄ peptide was added to 5 M Urea refolding buffer (5 M Urea, 0.4 M Arg, 100 mM Tris, 5 mM EDTA, 0.5 mM oxidized glutathione, 5 mM reduced glutathione), and then solubilized inclusion bodies were slowly added to 5 M Urea refolding buffer including a peptide. The refolding mixture was dialyzed several times against 10 mM Tris-HCl buffer. The refolded pMHC was purified with DEAE-Cellulose (ChemCruz), followed by MonoQ (GE Healthcare) and Superdex 200 Increase (GE Healthcare) columns. Biotinylation of refolded pMHC and its conformation were performed in the same manner as described above.

Before each round of selection, a small sample of yeast (˜1×10⁶ cells) was stained with tetramer pMHC and an anti-HA tag 6E2 antibody AlexaFluor 488 conjugate (Cell Signaling) for 1 hr on ice. For tetramer pMHC staining, biotinylated peptide/HLA-A*0201 for each target was incubated with streptavidin coupled to AlexaFluor 647. After washing cells with PBS-M (PBS, 0.5% BSA, and 1 mM EDTA) twice, cells were analyzed using Accuri C6 flow cytometer (BD Biosciences). MART1₂₆₋₃₅ (A2L)/HLA-A*0201 was used as a negative control for screening of NY-ESO1₁₅₇₋₁₆₅/HLA-A*0201, and NY-ESO1₁₅₇₋₁₆₅/HLA-A*0201was used for screening of MART1₂₆₋₃₅ (A2L)/HLA-A*0201. P53₆₅₋₇₃ (R9V). HLA-A*0201 was used as a negative control for screening of gp100₂₀₉₋₂₁₈ (T2M)/HLA-A*0201, gp100₂₈₀₋₂₈₈ (A9V)/HLA-A*0201, and KRAS₅₋₁₄/HLA-A*0201.

For the first round of selection, approximately 2×10⁹ cells were incubated with 250 μL Streptavidin Microbeads (Miltenyi Biotec) for 1 hr at 4° C. Cells passed through an LS column (Miltenyi Biotec) to remove nonspecifically binding cells. Then, 400 nM of biotinylated pMHC coupled to 250 μL of Streptavidin Microbeads was incubated with cells for 3-4 hr at 4° C. The pMHC-binding yeasts were collected via an LS column, washed with PBS-M, and then re-cultured in SD-CAA at 30° C. for 1-2 days. Yeasts were induced by SG-CAA at 20° C. for 2-3 days. Second round of selection was performed in the same manner, but with reduced amount of Streptavidin Microbeads (50 μL). Before third and fourth rounds of selection, negative screenings with each negative control pMHC were performed. At fourth round of selection, 40 nM of biotinylated pMHC was used for positive selection.

A small amount of yeast clones expressing TCRm scFv were incubated with 10-fold serially diluted pMHCs for 1 hr on ice. After washing cells with PBS-M, cells were stained with streptavidin coupled to AlexaFluor 647 for 15 min on ice and washed. Cells were analyzed by Accuri C6 flow cytometer. Assays were performed in biological triplicates.

Cells were stained with 100 nM of tetramer target peptide/HLA-A*0201 or 1,000 nM of tetramer non-target peptide/HLA-A*0201 for 2 hr on ice. After washing cells with PBS-M, cells were analyzed by Accuri C6 flow cytometer. Assays were performed in biological duplicates.

BIAcore T100 (GE Healthcare) was used to measure the K_{D} by single cycle kinetics method. The biotinylated pMHC was immobilized on a SA chip at 10-100 response unit (RU). Purified samples (scFv and IgG) were captured by injection of varying concentration (0.008-200 nM) with HBS-P⁺ for 120 see at a flow of 30 μL/min, then dissociation measured for 300 see with buffer flow. The signal of reference cells was subtracted from the measurements. Data analysis was performed using BIAcore T100 evaluation software.

Method for Expression and Purification of scFv and IgG. The candidate scFvs, positive control scFvs (WT and T1; Proc Natl Acad Sci USA. 2009 Apr. 7; 106(14):5784-8. doi: 10.1073/pnas.0901425106), and the candidate IgGs were expressed in Expi293 cells, and then purified with Ni-NTA resin or proteinA Sepharose FF resin.

Expi293 cells were transduced with the expression vector for single chain MART1₂₆₋₃₅ (A2L)/HLA-A*0201 with the transmembrane domain. Two days after transfection, 10-fold dilutions of purified IgG were incubated with 10⁵ cells for 2 hr. For peptide pulsed-cells assay, serial dilutions of MART1₂₆₋₃₅ (A2L) peptide were pulsed to A375 human melanoma cells for 4 hr at 37° C., and then 10 μg/mL of the purified IgG was incubated with cells for 2 hr. After staining cells with IgG, cells were washed and then incubated with proteinG AlexaFluor 488 conjugated for 1 hr. After washing cells with PBS-M, cells were analyzed by Accuri C6 flow cytometer (BD Biosciences). Assays were performed in biological triplicates.

Method for isolation of PBMCs. Peripheral blood mononuclear cells (PBMCs) were obtained from the Stanford Blood Bank. Cells in deidentified leukoreduction chambers from healthy platelet donors were processed as soon as possible and no later than 18 h after plateletpheresis.

Method for ADCC assay. 10⁶ cells/mL of A375 melanoma cells were incubated with 2 uM of CFSE for 10 min at 37° C. Cells were washed with PBS, and then 100 μM of MART1₂₆₋₃₅ (A2L) peptide or NY-ESO1₁₅₇₋₁₆₅ as a negative control was pulsed to cells at 37° C. After 4 hr incubation, cells were incubated with or without purified IgG. Subsequently, PBMCs were incubated as effector cells with tumor cells at an E:T ratio of 20:1 overnight at 37° C. After incubation with effector cells, cells were stained with PI and then analyzed by CytoFLEX Flow Cytometer. CFSE and PI double positive cells represented tumor target cells killed by ADCC. Data are mean±SD.

### Complete Variable Region Sequences:

Variable region of heavy chain for
anti-OVA/H2-K^{b} (PDB; 3cvh)
(SEQ ID NO: 84)
EVLLQQSGPELVKPGASVKIPCKASGYTFTDYNMDWVKQSHGKSLEWIG
DINPNNGGTIYNQKFKGKATLTVDKSSSAAYMEVRSLTSEDTAVYYCAR
KPYYGNFAWFAYWGQGTLVTVSA
Variable region of light chain for
anti-OVA/H2-K^{b} (PDB; 3cvh)
(SEQ ID NO: 85)
DIQVTQSSSSFSVSLGDRVTITCKASEDIYNRLAWYQQKPGNAPRLLIS
GATSLETGVPDRFSGSGSRKDYTLIITSLQTEDVATYYCQQYWSTPLTF
GAGTKLELK
Variable region of heavy chain for
anti-NY-ESO1/HLA-A*02: 01 (PDB; 3gjf)
(SEQ ID NO: 86)
EVQLLESGGGLVQPGGSLRLSCAASGFTFSTYQMSWVRQAPGKGLEWVS
GIVSSGGSTAYADSVKGRFTISRDNSKNTLYLQMNSLRAEDTAVYYCAG
ELLPYYGMDVWGQGTTVTVSS
Variable region of light chain for
anti-NY-ESO1/HLA-A*02: 01 (PDB; 3gjf)
(SEQ ID NO: 87)
QSELTQPRSVSGSPGQSVTISCTGTSRDVGGYNYVSWYQQHPGKAPKLI
IHDVIERSSGVPDRFSGSKSGNTASLTISGLQAEDEADYYCWSFAGSYY
VFGTGTDVTVL

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of designing an antigen binding region (ABR) that specifically binds to an MHC-peptide complex of interest, the method comprising:
identifying by structural analysis the peptide binding contact residues in an initial ABR;
generating a diverse ABR library of polynucleotide coding sequences by randomizing one or more codons encoding peptide binding contact residues in the initial ABR, optionally wherein from 7-10 contact residues are randomized;
expressing the library where the library ABR proteins are present on the cell surface; and
selecting for proteins that bind to the MHC-peptide complex of interest.

2. The method of claim 1, wherein the peptide of interest is distinct from the initial peptide and comprises at least 10⁸ different ABR sequences.

3-4. (canceled)

5. The method of claim 1, wherein the ABR is formatted as a single chain variable region (scFv).

6. The method of claim 1, wherein the MHC protein is a human Class I protein and the peptide is a viral or cancer neoantiqen peptide.

7. (canceled)

8. The method of claim 1, wherein the ABR comprises the CDR sequences SEQ ID NO:1 (H-CDR1); SEQ ID NO:2 (H-CDR2); and SEQ ID NO:5 (L-CDR2) appropriately combined with randomized CDR sequences SEQ ID NO:7 (H-CDR3); SEQ ID NO:8 (L-CDR1); and SEQ ID NO:9 (L-CDR3) and optionally the framework sequences of SEQ ID NO:86 and SEQ ID NO:88.

9. (canceled)

10. The method of claim 1, wherein the ABR comprises the CDR sequences SEQ ID NO:40 (L-CDR1); and SEQ ID NO:41 (L-CDR2) appropriately combined with randomized CDR sequences SEQ ID NO:43 (H-CDR1), SEQ ID NO:44 (H-CDR2), SEQ ID NO:45 (H-CDR3) and SEQ ID NO:46 (L-CDR3) and optionally the framework sequences of SEQ ID NO:84 and SEQ ID NO:85

11. (canceled)

12. A polynucleotide library comprising a diverse library of ABR sequences encoding CDR sequences SEQ ID NO:1 (H-CDR1); SEQ ID NO:2 (H-CDR2); and
SEQ ID NO:5 (L-CDR2) appropriately combined with randomized CDR sequences SEQ ID NO:7 (H-CDR3); SEQ ID NO:8 (L-CDR1); and SEQ ID NO:9 (L-CDR3) and optionally the framework sequences of SEQ ID NO:86 and SEQ ID NO:88; or
encoding CDR sequences SEQ ID NO:40 (L-CDR1); and SEQ ID NO:41 (L-CDR2) appropriately combined with randomized CDR sequences SEQ ID NO:43 (H-CDR1), SEQ ID NO:44 (H-CDR2), SEQ ID NO:45 (H-CDR3) and SEQ ID NO:46 (L-CDR3) optionally comprising the framework sequences of SEQ ID NO:84 and SEQ ID NO:85.

13-15. (canceled)

16. A population of cell comprising the library of claim 12.

17. An ABR that specifically binds to an MHC-peptide complex of interest produced by the method of claim 1.

18. An ABR where, the ABR comprises:
CDR sequences SEQ ID NO:1 (H-CDR1); SEQ ID NO:2 (H-CDR2); and SEQ ID NO:5 (L-CDR2) appropriately combined with affinity-selected CDR sequences selected from SEQ ID NO:10-12; SEQ ID NO:10, 4 and −15; SEQ ID NO:16-18; or.
CDR sequences SEQ ID NO:1 (H-CDR1); SEQ ID NO:2 (H-CDR2); and SEQ ID NO:5 (L-CDR2) appropriately combined with affinity-selected CDR sequences selected from SEQ ID NO:10-12; SEQ ID NO:10, 4 and −15; SEQ ID NO:16-18; or
CDR sequences SEQ ID NO:1 (H-CDR1); SEQ ID NO:2 (H-CDR2); and SEQ ID NO:5 (L-CDR2) appropriately combined with affinity-selected CDR sequences selected from SEQ ID NO:10 and 26-27; or
CDR sequences SEQ ID NO:1 (H-CDR1); SEQ ID NO:2 (H-CDR2); and SEQ ID NO:5 (L-CDR2) appropriately combined with affinity-selected CDR sequences selected from SEQ ID NO:10 and 29-30; or
CDR sequences SEQ ID NO:1 (H-CDR1); SEQ ID NO:2 (H-CDR2); and SEQ ID NO:5 (L-CDR2) appropriately combined with affinity-selected CDR sequences selected from SEQ ID NO:19 and 32-33; or SEQ ID NO:19 and 35-36; or
CDR sequences SEQ ID NO:40 (L-CDR1); and SEQ ID NO:41 (L-CDR2) appropriately combined with affinity-selected CDR sequences selected from SEQ ID NO:47-50; SEQ ID NO:51-54, SEQ ID NO:55-58 or SEQ ID NO:55 and 61-62.

19-23. (canceled)

24. The ABR of claim 18, operably linked to an effector polypeptide.

25. The ABR of claim 24, wherein the effector polypeptide is an Fc sequence, a chimeric antigen receptor, or a CD3-based bispecific T-cell engager.

26. A method of treating an individual for cancer, comprising administering an effective dose of an ABR of claim 24 to a patient in need thereof.


SEQUENCE LISTING (90 sequences)

SEQ ID NO: 1 - protein, 5 aa, Homo sapiens
TYQMS

SEQ ID NO: 2 - protein, 17 aa, Homo sapiens
GIVSSGGSTAYADSVKG

SEQ ID NO: 3 - protein, 10 aa, Homo sapiens
ELLPYYGMDV

SEQ ID NO: 4 - protein, 14 aa, Homo sapiens
TGTSRDVGGYNYVS

SEQ ID NO: 5 - protein, 7 aa, Homo sapiens
DVIERSS

SEQ ID NO: 6 - protein, 9 aa, Homo sapiens
WSFAGSYYV

SEQ ID NO: 7 - protein, 10 aa, Homo sapiens
  VARIANT (5)..(5): Where Xaa is an amino acid other than cysteine
ELLPXYGMDY

SEQ ID NO: 8 - protein, 14 aa, Homo sapiens
  VARIANT (4)..(4): Where Xaa is an amino acid other than cysteine
  VARIANT (10)..(10): Where Xaa is an amino acid other than cysteine
TGTXRDVGGXNYVS

SEQ ID NO: 9 - protein, 9 aa, Homo sapiens
  VARIANT (3)..(3): Where Xaa is an amino acid other than cysteine
  VARIANT (5)..(5): Where Xaa is an amino acid other than cysteine
  VARIANT (6)..(6): Where Xaa is an amino acid other than cysteine
  VARIANT (8)..(8): Where Xaa is an amino acid other than cysteine
WSXAXXYXV

SEQ ID NO: 10 - protein, 10 aa, Homo sapiens
ELLPYYGMDY

SEQ ID NO: 11 - protein, 14 aa, Homo sapiens
TGTNRDVGGYNYVS

SEQ ID NO: 12 - protein, 9 aa, Homo sapiens
WSIAQTYGV

SEQ ID NO: 13-14 - skipped (2 entries)

SEQ ID NO: 15 - protein, 9 aa, Homo sapiens
WSAANVYGV

SEQ ID NO: 16 - protein, 10 aa, Homo sapiens
ELLPFYGMDY

SEQ ID NO: 17 - protein, 14 aa, Homo sapiens
TGTLRDVGGYNYVS

SEQ ID NO: 18 - protein, 9 aa, Homo sapiens
WSAAMAYGV

SEQ ID NO: 19 - protein, 10 aa, Homo sapiens
ELLPGYGMDY

SEQ ID NO: 20 - protein, 14 aa, Homo sapiens
TGTDRDVGGQNYVS

SEQ ID NO: 21 - protein, 9 aa, Homo sapiens
WSPAWPYAV

SEQ ID NO: 22 - protein, 10 aa, Homo sapiens
ELLPSYGMDY

SEQ ID NO: 23 - protein, 14 aa, Homo sapiens
TGTDRDVGGYNYVS

SEQ ID NO: 24 - protein, 9 aa, Homo sapiens
WSPAWPYSV

SEQ ID NO: 25 - skipped

SEQ ID NO: 26 - protein, 14 aa, Homo sapiens
TGTDRDVGGKNYVS

SEQ ID NO: 27 - protein, 9 aa, Homo sapiens
WSKAQIYWV

SEQ ID NO: 28 - skipped

SEQ ID NO: 29 - protein, 14 aa, Homo sapiens
TGTLRDVGGLNYVS

SEQ ID NO: 30 - protein, 9 aa, Homo sapiens
WSWAPWYYV

SEQ ID NO: 31 - skipped

SEQ ID NO: 32 - protein, 14 aa, Homo sapiens
TGTRRDVGGSNYVS

SEQ ID NO: 33 - protein, 9 aa, Homo sapiens
WSMADLYTV

SEQ ID NO: 34 - skipped

SEQ ID NO: 35 - protein, 14 aa, Homo sapiens
TGTMRDVGGHNYVS

SEQ ID NO: 36 - protein, 9 aa, Homo sapiens
WSLADLYEV

SEQ ID NO: 37 - protein, 5 aa, Homo sapiens
DYNMD

SEQ ID NO: 38 - protein, 17 aa, Homo sapiens
DINPNNGGTIYNQKFKG

SEQ ID NO: 39 - protein, 12 aa, Homo sapiens
KPYYGNFAWFAY

SEQ ID NO: 40 - protein, 11 aa, Homo sapiens
KASEDIYNRLA

SEQ ID NO: 41 - protein, 7 aa, Homo sapiens
GATSLET

SEQ ID NO: 42 - protein, 9 aa, Homo sapiens
QQYWSTPLT

SEQ ID NO: 43 - protein, 5 aa, Homo sapiens
  VARIANT (3)..(3): Where Xaa is an amino acid other than cysteine
DYXMD

SEQ ID NO: 44 - protein, 17 aa, Homo sapiens
  VARIANT (1)..(1): Where Xaa is an amino acid other than cysteine
  VARIANT (10)..(10): Where Xaa is an amino acid other than cysteine
XINPNNGGTXYNQKFKG

SEQ ID NO: 45 - protein, 12 aa, Homo sapiens
  VARIANT (4)..(4): Where Xaa is an amino acid other than cysteine
  VARIANT (6)..(6): Where Xaa is an amino acid other than cysteine
  VARIANT (7)..(7): Where Xaa is an amino acid other than cysteine
KPYXGXXAWFAY

SEQ ID NO: 46 - protein, 9 aa, Homo sapiens
  VARIANT (4)..(4): Where Xaa is an amino acid other than cysteine
  VARIANT (5)..(5): Where Xaa is an amino acid other than cysteine
  VARIANT (6)..(6): Where Xaa is an amino acid other than cysteine
QQYXXXPLT

SEQ ID NO: 47 - protein, 5 aa, Homo sapiens
DYEMD

SEQ ID NO: 48 - protein, 17 aa, Homo sapiens
SINPNNGGTMYNQKFKG

SEQ ID NO: 49 - protein, 12 aa, Homo sapiens
KPYYGMNAWFAY

SEQ ID NO: 50 - protein, 9 aa, Homo sapiens
QQYYALPLT

SEQ ID NO: 51 - protein, 5 aa, Homo sapiens
DYDMD

SEQ ID NO: 52 - protein, 17 aa, Homo sapiens
SINPNNGGTIYNQKFKG

SEQ ID NO: 53 - protein, 12 aa, Homo sapiens
KPYRGFHAWFAY

SEQ ID NO: 54 - protein, 9 aa, Homo sapiens
QQYYGYPLT

SEQ ID NO: 55 - protein, 5 aa, Homo sapiens
DYYMD

SEQ ID NO: 56 - protein, 17 aa, Homo sapiens
LINPNNGGTVYNQKFKG

SEQ ID NO: 57 - protein, 12 aa, Homo sapiens
KPYTGHPAWFAY

SEQ ID NO: 58 - protein, 9 aa, Homo sapiens
QQYYAFPLT

SEQ ID NO: 59 - skipped

SEQ ID NO: 60 - protein, 17 aa, Homo sapiens
TINPNNGGTVYNQKFKG

SEQ ID NO: 61 - protein, 12 aa, Homo sapiens
KPYSGHPAWFAY

SEQ ID NO: 62 - protein, 9 aa, Homo sapiens
QQYYAFPLT

SEQ ID NO: 63 - DNA, 46 bp, Artificial sequence
  synthetic sequence
atgcagttacttcgctgtttttcaatattttctgttattgctagcg

SEQ ID NO: 64 - DNA, 32 bp, Artificial sequence
  synthetic sequence
gctgacagtaataagtggctacatcctccgtc

SEQ ID NO: 65 - DNA, 80 bp, Artificial sequence
  synthetic sequence
  misc_feature (38)..(46): n is a, c, g, or t
gacggaggatgtagccacttattactgtcagcaatacnnnnnnnnnccgctgactttcggggctgggacaaagctagagc

SEQ ID NO: 66 - DNA, 174 bp, Artificial sequence
  synthetic sequence
cgctgactttcggggctgggacaaagctagagcttaaaggcggaggtggatcaggtggaagtggcgggggagggggcggaagcgaagtattattacaacagtcaggccccgaattagtgaaaccaggagcttctgtcaagatcccgtgcaaggcaagcgggtatacttttaccg

SEQ ID NO: 67 - DNA, 147 bp, Artificial sequence
  synthetic sequence
  misc_feature (37)..(39): n is a, c, g, or t
  misc_feature (88)..(90): n is a, c, g, or t
  misc_feature (115)..(117): n is a, c, g, or t
ccgtgcaaggcaagcgggtatacttttaccgattatnnnatggattgggttaaacaaagccatggcaaatcattggaatggataggannnattaatcccaataacggaggcactnnntacaatcaaaagttcaaaggtaaggctacc

SEQ ID NO: 68 - DNA, 121 bp, Artificial sequence
  synthetic sequence
caatcaaaagttcaaaggtaaggctaccttgacggtggataagagttcatcagccgcatacatggaggtgaggtcccttacatccgaggacaccgctgtatactactgtgctaggaaaccg

SEQ ID NO: 69 - DNA, 98 bp, Artificial sequence
  synthetic sequence
  misc_feature (33)..(35): n is a, c, g, or t
  misc_feature (39)..(44): n is a, c, g, or t
ccgctgtatactactgtgctaggaaaccgtatnnngggnnnnnngcatggttcgcctactggggtcaggggactctagtcactgtatcagcgggatcc

SEQ ID NO: 70 - DNA, 52 bp, Artificial sequence
  synthetic sequence
cagcgtagtctggaacgtcgtatgggtaggatcccgctgatacagtgactag

SEQ ID NO: 71 - skipped

SEQ ID NO: 72 - DNA, 39 bp, Artificial sequence
  synthetic sequence
caacagctcccccgcacagtagtaaacggcagtatcctc

SEQ ID NO: 73 - DNA, 93 bp, Artificial sequence
  synthetic sequence
  misc_feature (44)..(46): n is a, c, g, or t
cgaggatactgccgtttactactgtgcgggggagctgttgcccnnntatggtatggacgtttggggacaaggcacgacggtcacggtgtcatc

SEQ ID NO: 74 - DNA, 156 bp, Artificial sequence
  synthetic sequence
ggacgtttggggacaaggcacgacggtcacggtgtcatctggaggaggtggttccggaggtggcggttccggcgggggaggtagccaatcagagctgacccagcctagaagtgtcagtggttcacccggtcaatccgtaactatttcttgcacagg

SEQ ID NO: 75 - DNA, 115 bp, Artificial sequence
  synthetic sequence
  misc_feature (47)..(49): n is a, c, g, or t
  misc_feature (65)..(67): n is a, c, g, or t
cagtggttcacccggtcaatccgtaactatttcttgcacaggcaccnnnagggacgtggggggannnaattacgtgtcctggtaccaacaacatccgggaaaagcccccaaattg

SEQ ID NO: 76 - DNA, 171 bp, Artificial sequence
  synthetic sequence
gtgtcctggtaccaacaacatccgggaaaagcccccaaattgataatccatgacgttattgaaagaagctcaggagtcccagacaggttcagcggtagcaagagtggcaataccgcaagcctgactatctctgggctgcaggccgaagacgaagcagactattattgctgg

SEQ ID NO: 77 - DNA, 128 bp, Artificial sequence
  synthetic sequence
  misc_feature (43)..(45): n is a, c, g, or t
  misc_feature (49)..(54): n is a, c, g, or t
  misc_feature (58)..(60): n is a, c, g, or t
gggctgcaggccgaagacgaagcagactattattgctggagtnnngcgnnnnnntatnnngtcttcgggacgggaacggacgtgaccgtcttaggatcctacccatacgacgttccagactacgctgg

SEQ ID NO: 78 - DNA, 31 bp, Artificial sequence
  synthetic sequence
ccagcgtagtctggaacgtcgtatgggtagg

SEQ ID NO: 79 - protein, 9 aa, Homo sapiens
SLLMWITQV

SEQ ID NO: 80 - protein, 10 aa, Homo sapiens
ELAGIGILTV

SEQ ID NO: 81 - protein, 9 aa, Homo sapiens
IMDQVPFSV

SEQ ID NO: 82 - protein, 9 aa, Homo sapiens
YLEPGPVTV

SEQ ID NO: 83 - protein, 10 aa, Homo sapiens
KLVVVGAGGV

SEQ ID NO: 84 - protein, 121 aa, Homo sapiens
EVLLQQSGPELVKPGASVKIPCKASGYTFTDYNMDWVKQSHGKSLEWIGDINPNNGGTIYNQKFKGKATLTVDKSSSAAYMEVRSLTSEDTAVYYCARKPYYGNFAWFAYWGQGTLVTVSA

SEQ ID NO: 85 - protein, 107 aa, Homo sapiens
DIQVTQSSSSFSVSLGDRVTITCKASEDIYNRLAWYQQKPGNAPRLLISGATSLETGVPDRFSGSGSRKDYTLIITSLQTEDVATYYCQQYWSTPLTFGAGTKLELK

SEQ ID NO: 86 - protein, 118 aa, Homo sapiens
EVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVS

SEQ ID NO: 87 - protein, 109 aa, Homo sapiens
QSELTQPRSVSGSPGQSVTISCTGTSRDVGGYNYVSWYQQHPGKAPKLIIHDVIERSSGVPDRFSGSKSGNTASLTISGLQAEDEADYYCWSFAGSYYVFGTGTDVTVL

SEQ ID NO: 88 - protein, 8 aa, Gallus gallus domesticus
SIINFEKL

SEQ ID NO: 89 - protein, 5 aa, Artificial sequence
  synthetic sequence
  VARIANT (1)..(5): Each copy of Gly Gly Gly Gly Ser at positions 1 to 5 is either present or absent such that the number of copies of Gly Gly Gl Gly Ser ranges from 1 to 5.
GGGGS

SEQ ID NO: 90 - protein, 9 aa, Homo sapiens
SVYDFFVWL